(19) United States
(12) Patent Application Publication
(10) Pub. No.: US 2011/0046079 A1
(43) Pub. Date: Feb. 24, 2011

(54) Use of Proton Pump Inhibitors as Drug Delivery Adjuvants

(76) Inventors: James M. Mullin, Havertown, PA (US); Giancarlo Mercogliano, Malvern, PA (US); James J. Thornton, Villanova, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/863,230

(22) PCT Filed: Jan. 16, 2009
(86) PCT No.: PCT/US09/31265

§ 371 Date: Nov. 2, 2010

Related U.S. Application Data
(60) Provisional application No. 61/021,479, filed on Jan. 16, 2008.

(51) Int. Cl. A61K 31/704; A61K 31/4439; A61P 9/04; A61P 9/06; A61P 25/08

(52) U.S. Cl. 514/26; 514/341

(57) ABSTRACT

Compositions and methods for drug delivery are disclosed.

This application claims priority under 35 U.S.C. §119(e) to U.S. Provisional Patent Application No. 61/021,479, filed on Jan. 16, 2008. The foregoing application is incorporated by reference herein.

### FIELD OF THE INVENTION

The present invention relates to the field of drug delivery. Specifically, methods using proton pump inhibitors as a drug delivery vehicle are disclosed.

### BACKGROUND OF THE INVENTION

Several publications and patent documents are cited throughout the specification in order to describe the state of the art to which this invention pertains. Each of these citations is incorporated herein by reference as though set forth in full.

Proton pump inhibitors (PPIs) are among the most widely prescribed and over-the-counter purchased drugs in the United States, with over 40 million Americans likely taking these drugs at any given time for heartburn and other upper gastrointestinal issues (Cappell, M. S. (2005) Med. Clin. North. Am., 89:243-91). The drugs have even come into use with infants where reflux disease is being more commonly suspected (Henry, S. M. (2004) Adv. Neonatal Care (2004) 4:235-47; Omari et al. (2007) J. Pediatr. Gastroenterol. Nutr., 44:41-4). Moreover, their use is very likely to be on the rise, as gastroesophageal reflux disease (GERD) is believed to be increasing in frequency. GERD is not simply “annoying heartburn” as it is believed to be the early stage of sequelae that will progress on to esophagitis, Barrett's esophagus, and esophageal adenocarcinoma in certain individuals (Maley and Rustgi (2006) J. Natl. Compr. Canc. Netw., 4:367-74). The final condition, esophageal adenocarcinoma, is in fact one of the most rapidly rising forms of cancer in the United States. Some reports contend that proper use of PPIs may inhibit the progression of those sequelae and thereby offer a protective effect regarding cancer of the esophagus (Lanas, A. (2005) Drugs, 65 Suppl 1:75-82).

### SUMMARY OF THE INVENTION

In accordance with the present invention, methods of delivering a therapeutic agent to a patient are provided. The methods comprise orally administering to a patient at least one composition comprising at least one proton pump inhibitor and/or at least one H2 antagonist, and at least one pharmaceutically acceptable carrier with, particularly prior to, orally administering at least one composition comprising at least one therapeutic agent and at least one pharmaceutically acceptable carrier. In a particular embodiment, the proton pump inhibitor is omeprazole or esomeprazole. In yet another embodiment, the therapeutic agent is selected from the group consisting of peptide, protein, nucleic acid molecule, oligonucleotide, chemical compound, and small molecule. In still another embodiment, the therapeutic agent is not indicated for oral administration and may be indicated for intravenous administration or administration by injection.

According to another aspect of the invention, compositions and kits are provided for practicing the instant invention.

### BRIEF DESCRIPTIONS OF THE DRAWING

FIG. 1 is a graph depicting the intrinsic variability in the sucrose permeability testing. A total of 10 healthy subjects and 10 patients with Barrett's esophagus were asked to perform three sucrose leak tests over a month period. The first two were 4 days apart. The third occurred thirty days after the first. Results shown represent the mean (±standard error) for each test for each test subject group. Within each subject group there were no statistical differences between tests (Student's t test, P>0.05).

FIG. 2 is a graph depicting the induction of an upper gastrointestinal (GI) leak after an eight week trial of PPIs. Sucrose permeability tests at the end of an 8 week course of NEXIUM® minus the corresponding GERD patient's permeability test before beginning NEXIUM® are shown for 26 patients. Each bar represents the result for one patient. Ascending bars indicate that the post-NEXIUM® leak was greater (21 of 26). Descending bars indicate that the post-NEXIUM® leak was smaller (4 of 25). Results shown are for the total amount of sucrose (mg) in an overnight urine sample as described hereinbelow. A total of 37 patients completed the 8 week course of therapy and both sucrose permeability tests, but the results of 11 were removed from consideration because the magnitude of their initial permeability test was >200 mg and suggested that an effect of a medication other than a PPI and/or a pathophysiological condition was complicating the interpretation.

FIG. 3 is a graph depicting the time course of the onset of the esomeprazole (NEXIUM®)-induced leak. The final minus the initial sucrose permeability test result for healthy test subjects placed on NEXIUM® (40 mg once/day) for a variable number of days. Ascending bars indicate that the post-NEXIUM® leak was greater. Descending bars indicate that the post-NEXIUM® leak was smaller. Each bar is the mean (±standard error) for the number of subjects shown. Double asterisks indicate statistical differences (P<0.05, Student's t test) between the post NEXIUM® and pre NEXIUM® test results for the NEXIUM® duration indicated. Significant differences existed for the 5, 7 and 9 day durations.

FIG. 4 is a graph depicting the reversibility of the esomeprazole-induced leak. Results are pooled for the subjects in FIG. 2 who demonstrated a significant difference between pre- and post-NEXIUM® permeability tests (the 5, 7 and 9 day subject groups). These subjects also performed a third and final sucrose permeability test 4 days after finishing their NEXIUM® course. The means of those three tests are shown (±standard error) (n=9). The first and third tests are not significantly different from each other. Both are significantly different from the middle or second permeability test (P<0.001, Student's t test).

FIG. 5A is a graph of a real-time trace of transepithelial resistance across two pieces of rat gastric corpus tissue from the same animal. Samples were treated with the acid secretagogue, dB-cAMP, and subsequently treated with either DMSO (control) or 200 μM omeprazole/DMSO. FIG. 5B is a real-time trace of short circuit current across two pieces of rat gastric corpus tissue from the same animal. Upon addition of omeprazole, there is slight stimulation of short circuit current, a phenomenon consistently observed in all experiments performed with 200 μM omeprazole. FIG. 5C is a graph of the pH drop in mucosal fluid bathing the same two pieces of rat gastric corpus tissue in FIG. 5A after addition of the acid secretagogue, dB-cAMP (pre-flux). 200 μM omeprazole, which was added to one piece of tissue during the flux at approximately 125 minutes running time, inhibited acid secretion and prevented further pH drop. FIG. 5D is a real-time trace of 0.1 mM ¹⁴C[-D-] mannitol flux across two segments of rat gastric corpus which were treated with dBcAMP. Results shown are the mean+/−standard error.

FIG. 6 is a graph of the dose-dependence of the PPI-induced permeability increase. A marked increase in transepithelial permeability of rat gastric corpus first occurred at a concentration of 25 μM omeprazole (with no significant effect from 1, 5, or 10 μM) and plateaued thereafter at concentrations of 100 μM and 200 μM. This dose-response also correlates with omeprazole's dose-dependent inhibition of acid secretion, which also plateaued at 25 μM omeprazole, where 100% inhibition of acid secretion (as defined by decrease in mucosal fluid pH) was observed. Results shown are the mean+/−standard error, for three experiments at each omeprazole concentration.

FIGS. 7A-7C depict thin-layer radiochromatography analysis of tritium crossing the rat gastric corpus mucosa after application of ³H-digoxin to the mucosal fluid compartment. The y-axis represents the amount of radioactivity (cpm) present at different distances from the origin on the chromatogram in a sample experiment. Serosal fluid bathing control tissue (FIG. 7A) and omeprazole-treated tissue (FIG. 7B) was analyzed at the end of the 135 minute flux period by silica gel thin-layer chromatography using an isopropanol/water (120:30) solvent in a sandwich apparatus, and sprayed with Kedde. Serosal fluid samples were evaporated to dryness in a Speed Vac to concentrate the radioactivity and resuspended in methanol prior to chromatography. The box represents the digoxin detected chemically after spraying with reagent. Two distinct radiolabeled species are identified in serosal saline samples, one of which co-migrates with unlabeled digoxin (FIG. 7C). Note that proportionally more serosal radioactivity is ³H-digoxin when omeprazole has induced a leak in the gastric barrier (FIG. 7B vs. FIG. 7A).

FIG. 8 provides a schematic of the flux of ³H-digoxin from the mucosal fluid compartment across the gastric mucosa into the serosal fluid compartment. Note the two possible radiolabeled species entering the serosal fluid compartment, either intact ³H-digoxin (without undergoing any metabolism as a result of permeating the gastric mucosa by a paracellular route) or ³H-metabolites (products of metabolized ³H-digoxin resulting from transit across the mucosa by a transcellular route).

FIGS. 9A and 9B provide the molecular structure of digoxin and phenytoin, respectively.

FIG. 10 is a graph of the gastric leak following a five day course of the H-2 blocker famotidine.

### DETAILED DESCRIPTION OF THE INVENTION

It has been recently shown that Barrett's esophagus patients manifest a transmucosal leak in their upper GI tract (Mullin et al. (2006) Dig. Dis. Sci., 51:2326-36). NSAIDs are also known to produce upper gastrointestinal transmucosal leak (Meddings et al. (1993) Gastroenterology, 104:1619-26; Smecuol et al. (2001) Gut, 49:650-5). However, NSAIDs are not a necessarily common chronic medication in patients with Barrett's esophagus. One emblematic medication for the Barrett's esophagus patient group is, of course, PPIs. As a group, these patients are generally diagnosed because their associated symptomatic GERD drove them to see a gastroenterologist and have an upper endoscopy performed. The symptomatic nature of their GERD would also have induced them to be on a PPI persistently.

Based on the previous understanding of PPIS, one would have expected that the effect of PPIs on upper gastrointestinal leak was that these medications would reduce leak, i.e. improve barrier function. They are well known to reduce gross esophageal inflammation or improve symptoms by reducing the acidity of the refluxed gastric contents (Kahrilas et al. (2000) Aliment. Pharmac. Therap., 14:1249-58). An increased transmucosal leak in esophagitis patients was observed previously (Mullin et al. (2006) Dig. Dis. Sci., 51:2326-36). It was reasoned that any reduction of macroscopic or microscopic inflammation/ulceration or the cellular/molecular changes seen in acid-reflux-associated dilation of intercellular spaces (Tobey et al. (1996) Gastroenter., 111:1200-5) in a first-time-presenting GERD patient as a result of an 8-week course of a PPI would only serve to improve upper GI barrier function.

As described herein, oral sucrose was used as a probe of upper GI permeability. A similar approach has been used to clinically evaluate leak in Barrett's esophagus. Sucrose, because it is a disaccharide, lacks a transport protein in any mammalian cell and can only pass through the upper GI barrier paracellularly. In other words, sucrose must find a pathway between cells—either through leaky tight junctions or through a frank break (e.g. ulceration) in the upper GI mucosa (Munck and Rasmussen (1977) J. Physiol., 271:473-88; Menzies, I. S. (1972) Clin. Sci., 42:18P). Since sucrose is hydrolyzed to glucose and fructose on the surface of the duodenum, its leak must be proximal to this point. Once in the bloodstream, sucrose cannot be reabsorbed by the kidney for similar reasons, and thus passes quantitatively into the urine (Meddings et al. (1995) Am. J. Ther., 2:843-849). Having test subjects drink a concentrated sucrose solution and collect an overnight urine sample for analysis of upper GI leak has been used in many clinical studies since it was first described (Meddings et al. (1993) Gastroenterology, 104:1619-26). As described hereinbelow, sucrose (upper GI) leak in various patient classes is studied both before PPI medication was begun and at the time a course of medication was being completed.

PPIs have not been reported to induce necrosis or apoptosis in cells in the stomach. Given the general safety profile of PPI medications and the near immediate onset of leak in animal models, it does not appear, without being bound by theory, that the PPI-induced leak is due to cell death in the gastric mucosa. Micron-size “holes” in the gastric barrier are also therefore unlikely. PPIs are, however, known to affect intracellular potassium and calcium homeostasis by inhibiting the H,K-ATPase and calcium homeostasis (Yenisehirli and Onur (2006) Pharmacol. Res., 54:397-405). PPIs have also been reported to have effects on the cellular cytoskeleton (Hotta et al. (1998) J. Cardiovasc. Pharmacol., 31:146-56; Aydin et al. (2003) J. Gastroenterol., 38:765-71; Rhoden, K. J. (2000) J. Pharm. Pharmacol., 52:857-62). Notably, there is a known, well-described interaction between the cytoskeleton and tight junctions (Fanning et al. (1998) J. Biol. Chem., 273:29745-53; Madara, J. L. (1987) Am. J. Physiol., 253:C171-5; Fasano et al. (1995) J. Clin. Invest., 96:710-20). A tight junction leak induced by PPIs would be consistent with the findings presented herein.

Tight junctions are known to exhibit remarkable size and charge selectivity in both basal and ‘stimulated’ states (Knipp et al. (1997) J. Pharm. Sci., 86:1105-10; Turner, J. R. (2006) Am. J. Pathol., 169:1901-9; Mullin et al. (1997) J. Cell. Physiol., 171:226-33). Here, it has been demonstrated that the uncharged, electrolytes D-mannitol (180 mw), sucrose (350 mw) and polyethyleneglycol (4000 mw) can pass through the PPI induced leak. Accordingly, it is possible that other medications/therapeutic agents that a patient may be taking in addition to PPIs, may have the kinetics of their appearance in the bloodstream altered by the (PPI) induction of a gastric leak pathway. Such a change in kinetics may have undesirable side effects not seen without concomitant PPI use. It may be especially problematic if a patient is taking a first drug for some time, then begins to simultaneously take PPIs as well, thereby opening up a leak pathway for the first drug that does not involve metabolic breakdown of the first drug and altering its rate of appearance in the bloodstream once titrations of the first drug may already have been performed. It is interesting in this regard that elevation in blood digoxin levels has been reported for some time with PPI use, with one recent case study reporting digoxin elevations over 300%, resulting in hospitalization (Welage and Berardi (1994) J. Pharm. Pract., 7:177-195; Humphries and Merritt (1999) Aliment Pharmacol. Ther., Suppl 3:18-26; Kiley et al. (2007) South Med. J., 100:400-2). This phenomenon will be even more problematic in an elderly patient population, where individuals on as many as 6 medications simultaneously are not uncommon. Notably, omeprazole has been known to alter the side effect profile and/or blood level of warfarin, cyclosporine, diazepam and phenytoin as well as digoxin, although the newer derivatives of omeprazole appear to have fewer drug-drug interactions than omeprazole itself (Humphries and Merritt (1999) Aliment Pharmacol. Ther., Suppl 3:18-26; Robinson and Horn (2003) Drugs, 63: 2739-2754). While it is possible that these interactions/side effects are the result of the well-described omeprazole-induced inhibition of cytochrome P450 degradation of these drugs—or even simply the result of altered intraluminal duodenal pH on these drug's absorption, the previously unrecognized leak phenomenon that is presented herein may also figure prominently.

The powerful mitogenic protein, EGF (epidermal growth factor) exists in saliva at its highest level in any fluid in the body, a concentration over 5,000× the level in blood (Gregory et. al. (1979) Gastroenterology, 77:313-8). This EGF constantly bathes and contacts the mucosal surface of the stomach. If PPIs open up a transmucosal leak to EGF (a relatively small protein at only 6 kDa), it would rapidly move down its concentration gradient into the gastric stroma. Here, it would encounter receptors on the back (basolateral) sides of the gastric epithelia (which it normally would never see) as well as receptors on stromal fibroblasts (see Mullin, J. M. (2004) Science STKE., 216:pe2-pe4). The result could then be persistently altered cell kinetics in this tissue. Certainly the well described gastric mucosal thickening and fundic polyposis that can often accompany PPI use, and are currently ascribed to elevated blood gastrin levels, could involve increased stromal compartment EGF coming from the lumen through a PPI-induced leak (Larsson et al. (1988) Gastroenterology, 95:1477-86; el-Zimaity et al. (1997) Am. J. Gastroenterol., 92:1858-60; Choudhry et al. (1998) Am. J. Clin. Pathol., 110:615-21). A PPI-induced paracellular leak to food antigens or bacterial toxins may have similar physiological downside.

However, it is important to note that PPIs are widely used and have been found to be safe, even over long periods of use. PPIs are relatively well-tolerated medications with very little reported clinical downside (Freston, J. W. (1997) Am. J. Gastroenterol., 92(4 Suppl):51S-5S). Indeed, it is probable that many PPI-induced leaks are real but harmless under most circumstances. Significantly, the PPI and H2 blocker induced leak allows only certain molecules to pass based on, for example, size, charge, and hydrophilicity. This is in contrast to other known leaks which are nonspecific.

The opening of a paracellular pathway by PPIs allows for a novel means of oral drug delivery, such as small molecules, oligonucleotides, peptides, and proteins. Indeed, the PPI-induced leak allows for the oral administration of drugs normally administered by injection or intravenous infusion. By definition a paracellular pathway, unlike a transcellular route, is free of the possibility of metabolic degradation of the drug in question. Therefore, the orally administered drug will arrive in the bloodstream structurally intact and biologically active. Co-administration of PPIs with a compound, protein, peptide and/or oligonucleotide allows for the passages of the compound, protein, peptide and/or oligonucleotide intact to the bloodstream prior to degradation in the GI tract, particularly the duodenal lumen.

### Definitions

The following definitions are provided to facilitate an understanding of the present invention:

“Pharmaceutically acceptable” indicates approval by a regulatory agency of the Federal or a state government or listed in the U.S. Pharmacopoeia or other generally recognized pharmacopoeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), water, aqueous solutions, oils, bulking substance (e.g., lactose, mannitol), excipient, auxilliary agent or vehicle with which an active agent of the present invention is administered. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3rd Ed.), American Pharmaceutical Association, Washington, 1999.

As used herein, “proton pump inhibitors” refer to compounds that block gastric acid secretion by inhibiting the H⁺/K⁺-ATPase enzyme system at the secretory surface of the gastric parietal cell. In a particular embodiment, proton pump inhibitors are substituted benzimidazoles and substituted azabenzimidazoles. Proton pump inhibitors include, without limitation, omeprazole (5-methoxy-2((4-methoxy-3,5-dimethyl-2-pyridinyl)methyl)-sulfinyl)-1H-benzimidazole, PRILOSEC®), esomeprazole (NEXIUM®), lansoprazole (2-(((3-methyl-4-(2,2,2-trifluoro-ethoxy)-2-pyridinyl)methyl)sulfinyl)-1H benzimidazole, PREVACID®), pantoprazole (5-(difluoromethoxy)-2-(((3,4-dimethoxy-2-pyridinyl)methyl)sulfinyl)-1H-benzimidazole), rabeprazole (2-(((4-(3-methoxypropoxy)-3-methyl-2-pyridinyl)methyl)sulfinyl)-1H-benzimidazole, ACIPHEX®), pariprazole, leminoprazole, timoprazole, tenatoprazole, disulprazole, RO 18-5362, IY 81149, and analogs thereof. In a particular embodiment, the PPI is omeprazole or esomeprazole.

As used herein, histamine 2 receptor antagonists (H₂ antagonists or H₂ blockers) are compounds that block H₂ receptors. In a particular embodiment, the H₂ antagonists of the instant invention include compounds which can be demonstrated to function as competitive or non-competitive inhibitors of histamine-mediated effects in those screening models specifically dependent upon H₂ receptor function, but lack significant histamine antagonist activity in those screening models dependent upon H₁ receptor function. Examples of H₂ antagonists include, without limitation, cimetidine (TAGAMET®), famotidine (PEPCID®), nizatidine (AXID®), ranitidine (ZANTAC®), ranitidine bismuth citrate (PYLORID®), ebrotidine, mifentidine, roxatidine, pisatidine and aceroxatidine.

As used herein, the terms “therapeutic agent” or “drug” refers to a compound which prevents, inhibits, or treats the symptoms of a particular disorder or disease. A “therapeutic agent” or “drug” can be, without limitation, a peptide, a protein (including antibodies), a nucleic acid molecule, an oligonucleotide, a chemical compound, a small molecule, or any other material.

As used herein, the term “small molecule” refers to a substance or compound that has a relatively low molecular weight (e.g., less than 2,000). Typically, small molecules are organic, but are not proteins, polypeptides, or nucleic acids.

As used herein, oral administration refers to the introduction of a drug or therapeutic agent into a subject by way of the oral cavity (e.g. in aqueous liquid or solid form). Oral administration also encompasses the ingestion of a drug or therapeutic agent by swallowing or chewing.

### Methods and Compositions

In accordance with the instant invention, methods are provided for the administration of at least one therapeutic agent to a patient in need thereof. Generally, the methods comprise administering at least one proton pump inhibitor (PPI) and/or at least one H₂ antagonist with the at least one therapeutic agent. In a particular embodiment, the method comprises administering at least one PPI and at least one therapeutic agent. Preferably, the proton pump inhibitor and/or H₂ antagonist and the therapeutic agent are administered orally and the PPI and/or H₂ antagonist is administered prior to the therapeutic agent. In a particular embodiment, the PPI and/or H₂ antagonist and therapeutic agent are not co-administered with an acid secretagogue.

In a preferred embodiment, the PPI and/or H₂ antagonist is administered long enough before the therapeutic agent so as to allow the blood levels of the PPI and/or H₂ antagonist to stabilize and/or peak. The PPI and/or H₂ antagonist may be administered concurrently with the therapeutic agent, preferably at least 1 or 2 days before, more preferably, at least 3 or 4 days before, and still more preferably 5 or more days before the therapeutic agent. When the therapeutic agent requires multiple doses or administrations, the PPI and/or H₂ antagonist may be administered prior to each administration of the therapeutic agent depending upon the specific PPI and/or H₂ antagonist used. Alternatively, if the PPI and/or H₂ antagonist levels remain high, then the therapeutic agent may be re-administered to the patient without re-administering the PPI and/or H₂ antagonist. In other words, the PPI and/or H₂ antagonist should be administered at intervals sufficient to maintain the upper GI leak and the therapeutic agent should be administered on its own schedule for effectiveness in preventing, inhibiting, or treating a particular disorder or disease. The appropriate interval for administration in a particular case would normally depend on the condition of the patient.

In a particular embodiment, the therapeutic agent is a peptide (e.g., an erythropoiesis-stimulating peptide such as HEMATIDE™, Affymax, Inc., Palo Alto, Calif.; and an antiarrhythmic peptide such as rotigaptide (ZP123, Ac-D-Tyr-D-Pro-D-Hyp-Gly-D-Ala-Gly-NH₂), Wyeth Pharmaceuticals, Madison, N.J.), a protein (e.g., insulin and epidermal growth factor), an antibody, a nucleic acid molecule (e.g., an anticancer nucleic acid molecule such as ANGIOZYME™ (a ribozyme which cleaves vascular endothelial growth factor receptor (VEGFR)-1 mRNA), RPI Pharmaceuticals, Santa Ana, Calif.), an oligonucleotide (e.g., siRNAs and antisense molecules (e.g., an antisense molecule for the treatment of high cholesterol and cardiovascular disease such as ISIS 301012 (which inhibits apoB-100), ISIS Pharmaceuticals, Carlsbad, Calif.), a chemical compound, or a small molecule.

The therapeutic agent can be of low molecular weight (less than about 2,000), medium molecular weight (about 2,000 to about 4,000), or high molecular weight (greater than about 4,000). In a particular embodiment, the therapeutic agent is at least medium molecular weight and, more preferably, is of a high molecular weight. In another embodiment, the therapeutic agent has a molecular weight less than about 10,000. In yet another embodiment, the therapeutic agent has a molecular weight less than about 4,000. In still another embodiment, the therapeutic agent has a molecular weight of at least about 500 and, more preferably, at least about 200. In a specific embodiment, the therapeutic agent has a molecular weight from about 200 to about 10,000.

In yet another embodiment, the therapeutic agent is hydrophilic as opposed to hydrophobic. As used herein, “hydrophilic” refers to a material that will dissolve or disperse in water at a temperature of 23° C. in an amount of at least 7% by weight, at least 10% by weight, at least 20% by weight, or at least 40% by weight, based on the total weight of the hydrophilic material and the water. As used herein, the term “hydrophobic” refers to a material that will not significantly dissolve in water at 23° C. This means that less than 5% by weight, less than 1% by weight, less than 0.5% by weight, or, particularly, less than 0.1% by weight, based on the total weight of the hydrophobic material and the water, will dissolve.

The drug delivery method of the instant invention allows for the delivery of a drug to the bloodstream without degradation. In a particular embodiment, the therapeutic agent is not indicated for oral administration or is deemed unsuitable for oral administration. The therapeutic agent may be of a size, shape, or charge that prevents it from being absorbed into the bloodstream through the GI tract barrier, thereby preventing it from being administered orally. The drug or therapeutic agent of the instant invention may not meaningfully traverse the gastric or intestinal epithelial barrier to the bloodstream. Alternatively, the drug or therapeutic agent may be degraded within GI tract to an unacceptable degree. In other words, the therapeutic agent does not have a gastrointestinal uptake pathway and/or is destroyed in the small bowel. The induced leak of the instant invention allows for the uptake of a therapeutic agent prior to degradation. In yet another embodiment, the therapeutic agent does not have a cellular uptake pathway. In a preferred embodiment, the therapeutic agent is ordinarily administered only by injection or intravenously.

The PPI and/or H₂ antagonist and therapeutic agent may be contained within a composition comprising at least one pharmaceutically acceptable carrier. The PPI and therapeutic agent may be contained in the same composition or may be contained within separate compositions. Oral compositions of the instant invention may be, for example, in pill form (e.g., capsule, tablet, and lozenge, optionally time-released), a solid, a powder, a solution, a syrup, an emulsion, a dispersion, a micelle, a liposome, or any other form suitable for use. Common carriers include, without limitation, water, oil, buffered saline, ethanol, polyol (for example, glycerol, propylene glycol, liquid polyethylene glycol and the like), dimethyl sulfoxide (DMSO), detergents, suspending agents, glucose, lactose, gum acacia, gelatin, mannitol, starch paste, magnesium trisilicate, talc, corn starch, keratin, colloidal silica, potato starch, urea, medium chain length triglycerides, dextrans, other carriers suitable for use in manufacturing preparations, in solid, semisolid, or liquid form, and suitable mixtures thereof. In addition excipients and auxiliary, stabilizing, preserving, thickening, flavoring, and coloring agents may be included in the compositions.

The composition(s) comprising the PPI and/or H₂ antagonist and therapeutic agent may be contained within a kit. In one embodiment, the kit may comprise at least one composition comprising a PPI(s) and/or H₂ antagonist(s) and a pharmaceutically acceptable carrier(s) and at least one composition comprising the therapeutic agent(s) and a pharmaceutically acceptable carrier(s). In another embodiment, the kit may comprise at least one composition comprising a PPI(s) and/or H₂ antagonist(s) and a pharmaceutically acceptable carrier(s) and at least a second composition comprising a pharmaceutically acceptable carrier for oral administration. The therapeutic agent may be added to the second composition (preferably a liquid) for administration by the practitioner.

The dose and dosage regimen of the compositions comprising the PPI and/or H₂ antagonist and/or therapeutic agent may be determined by a physician considering the patient's age, sex, weight, general medical condition, and the specific condition and severity thereof for which the preparation is being administered. A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Appropriate concentrations for alleviation of a particular pathological condition may be determined by dosage concentration curve calculations, as known in the art.

In accordance with the present invention, the appropriate dosage for the administration of the PPI and/or H₂ antagonist may be the dosage currently used for the treatment of heartburn and/or other upper GI issues. The dosage of the composition(s) comprising the therapeutic agent may be determined by evaluating the efficiency by which the therapeutic agent enters the blood stream through the PPI and/or H₂ antagonist induced upper GI leak. Animal models may be used to gauge the ability of the therapeutic agent(s) to traverse the PPI and/or H₂ antagonist induced GI leak.

The following examples provide illustrative methods of practicing the instant invention, and are not intended to limit the scope of the invention in any way.

### Example 1

### Evidence of a PPI Gastric Leak

### Recruitment

Healthy controls were recruited without regard to gender or ethnicity, from an age range of 18-80. Their exclusion criteria were no prior gastrointestinal surgery and no current gastrointestinal disease. Because of the nature of the permeability test being used, diabetics and those with renal insufficiency were likewise excluded. Those participating did so only after personally signing an informed consent that was approved prior by the Lankenau Hospital Institutional Review Board.

Patients with known Barrett's esophagus were recruited from a participating gastroenterology practice (Main Line Gastroenterology Associates). Their Barrett's diagnosis was made after upper endoscopy and biopsy histology (goblet cell metaplasia). With the exception of their Barrett's esophagus and associated GERD, these patients met the above criteria and were recruited in the same manner. These patients were all on chronic PPI medication at the time of their permeability tests.

Previous work has shown that GERD without mucosal abnormalities (as evidenced in previous endoscopic examination) did not result in a mucosal leak to sucrose (Mullin et al. (2006) Dig. Dis. Sci., 51:2326-36). Those GERD patients, unlike those in part of the instant study, had long-standing GERD and were already under the medical care of a gastroenterologist before entering the study.

Patients with GERD were recruited from a primary care practice, to which they were presenting for the first time with their disease. These patients were either PPI- and H-2 blocker-naïve or had not taken such medications for at least a 30 day period (though simple antacids were taken in that time frame). With the exception of their GERD, these patients met the above criteria at the time of their recruitment, and were recruited in a similar manner. Several days after performing their initial sucrose permeability test these patients—who had not been previously endoscoped—were given an upper endoscopy examination at study expense. Certain patients were then found to have Barrett's esophagus, esophagitis, gastritis or ulcerative disease though the majority did not manifest any gross abnormalities.

### Sucrose Permeability Test

All patients and healthy controls consumed in their homes a chilled solution of 100 g of sucrose in 200 cc of water containing 5 g of a citric acid-based flavoring agent at bedtime. Patients undergoing upper endoscopy had their exams performed only after the first sucrose test was completed (and at least six weeks before the second test) so as not to have scope trauma or biopsies interfere with permeability measurement. An 8 hour overnight urine sample was collected in a container with 5 ml of 10% thymol in isopropanol as antimicrobial preservative. A courier service picked up urine containers at the patient's home and delivered them to the research laboratory. Total urine volume and refractive index were measured and recorded. The concentration of sucrose in the urine sample was then measured by an enzymatic/spectrophotometric assay after prior desalting of the urine sample by anion and cation exchange resins (Mullin et al. (2006) Dig. Dis. Sci., 51:2326-36; Meddings et al. (1993) Gastroenter., 104:1619-26). Total amount of sucrose in the urine in mg was determined by multiplying the urine volume in ml by the sucrose concentration in mg/ml. This amount of sucrose equates to the amount of sucrose which leaked from the upper GI lumen.

### Materials

The sucrose solutions were a generous gift of Perk Scientific, Inc. (Yeadon, Pa.). Enzymatic reagents for determination of urine glucose and sucrose concentrations (invertase, hexokinase, and glucose-6-phosphate dehydrogenase) as well as cofactors (ATP and NADP) were products of Sigma Aldrich Chemical Co.

### Statistics

In comparing pre- and post-PPI sucrose leak levels, a paired Student's t test was used. In evaluating sucrose leak levels before, during and after PPI use, an ANOVA was utilized.

### Results

In order to first assess the intrinsic reproducibility of the sucrose permeability test, two groups of volunteers were asked to take the test three times: twice at a three day interval, and a third time thirty days later. The first group was a disease-free healthy control group and the second group was an already diagnosed Barrett's esophagus group as defined above. Their demographic criteria are described in Table 1. All urine samples were aliquoted, stored at −70° C. and assayed at the same time, as described above.

Demographic criteria for this study's GERD population are shown in Table 1. Whether a correlation existed between the age of patients versus their initial SPT values or versus the magnitude of difference between their pre-esompreazole and postesomeprazole sucrose leaks was studied. Neither parameter correlated with age. Similarly there was no correlation of either leak parameter with tobacco use, gender, nor with ethnicity (although the percentage of Asians in the study was too low to test for a meaningful correlation with this group). Correlations were likewise not observed between either leak parameter or endoscopic findings. This was even true for the GERD patients who were found to have esophageal columnar metaplasia or esophageal Goblet cell metaplasia. This finding is in partial conflict with the earlier findings regarding Barrett's esophagus patients (Mullin et al. (2006) Dig. Dis. Sci., 51:2326-36). The explanation may be that this was performed with previously endoscoped and diagnosed Barrett's patients who were under (PPI) therapy for their reflux symptoms while this current study's de-novo—diagnosed Barrett's population was not undergoing therapy for their reflux until after their initial sucrose leak test.

TABLE 1
Patient demographics.

| | Age | Percent | Percent |
|---|---|---|---|
| | Range | Male | Caucasian |
| GERD Population (26) | 24-70 | 48% | 64% |
| | [48] | | |
| Barrett's Population (8) | 47-81 | 50% | 100% |
| | [63] | | |
| Non-disease group (34) | 21-67 | 62% | 82% |
| | [36] | | |

The number in parenthesis represents the number of patients in that group. The number in brackets is the mean age of that group. The non-disease group comprises both the short term NEXIUM ® and the Multiple Sucrose Test studies.

Although test subjects are asked to refrain from NSAIDs and alcohol the day of their permeability test and to refrain from eating or drinking after consuming the sucrose test solution until the following morning, numerous other sources of variability and error present themselves. The following potential effects on the outcome of the sucrose leak test were not controlled for: the effect of foods eaten within the previous 6 hours; other medications (with unknown potential effects) that a patient may be taking; the patient's posture after drinking the sucrose test solution (supine, sitting or erect), menstrual cycle, exercise, allergies, transient minor disease such as viral infections, and of course, compliance and ability to follow test directions.

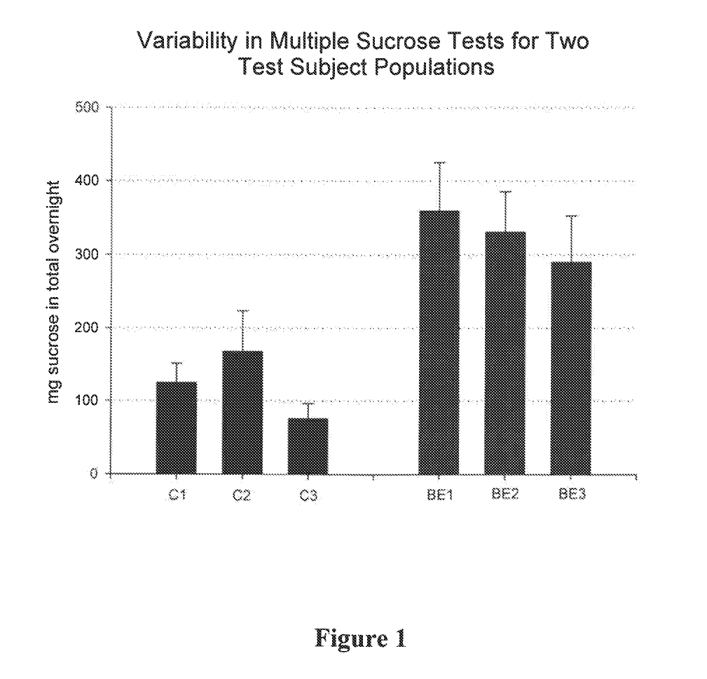

As shown in FIG. 1, the Barrett's group in general showed a significantly and dramatically greater sucrose leak than the control group at each and every test (P<0.01). In other words the variability observed in taking the test at different times never overrode the disease-related effect that the test was able to discern. In the control group, though individual differences as great as 50% were observed between the mean result of the second permeability test vs. the third permeability test, the differences did not achieve statistical significance. For the disease group, namely patients with a prior diagnosis of Barrett's esophagus, the variability in the three different sucrose permeability tests was noticeably less, the range of the means being less than 20%, and again no significant differences existing between the three test times. The largest standard error observed (as a percent of the mean) was 34% for the second test performed by the control group. The standard errors for the three tests performed by the Barrett's patients were a remarkably concise 18%, 16% and 21% of their respective means. If one examines standard error collectively for all three tests it was 22% of the collective mean for non-disease controls and 34% of the collective mean for Barrett's patients. Therefore, it would be predicted that under these conditions the sucrose permeability test can detect an induced leak, either pharmacological- or disease-based, if the change in permeability is greater than 60% of baseline permeability and the population size is at least moderate (n>20). These current results were obtained with a small population (n=10) and without the assignation and removal of outliers. The implementation of greater control over the sources of potential variability named above (e.g. regimenting the subject's posture for 20 minutes after drinking the test solution or defining the period of fast before taking the test) and with the use of outlier identifying protocols, one would have still lower variability and, hence, greater ability to effectively screen for altered barrier function in reasonable-sized populations.

The next study posed the question of whether PPI therapy would improve barrier function in the upper GI tract by allowing for reduction of mucosal inflammation and damage ensuing from esophagitis and gastritis. An early hypothesis was that PPI therapy would improve barrier function in the upper GI tract of first-time-presenting GERD patients, by allowing for reduction of mucosal inflammation and damage ensuing from any esophagitis or gastritis. First-time-presenting GERD patients who were either PPI- and H-2 blocker-naïve—or refraining from those medications for at least 30 days—were recruited for this study. As previously mentioned, this was in contrast to an earlier study (Mullin et al. (2006) Dig. Dis. Sci., 51:2326-36) which utilized GERD patients who had been previously diagnosed and under the care of a gastroenterologist. Patients were asked to perform a first sucrose permeability test before beginning an 8 week course of esomeprazole (NEXIUM®) at a dose of 40 mg/day. 3-5 days after beginning their medication, an upper endoscopy exam was performed to assess the condition of their upper duodenum, stomach and esophagus.

A 4 week follow up check was performed on all patients, and all reported significant symptom improvement with mild if any side effects. Within three days before their medication course was completed, all patients performed a second and final sucrose permeability test.

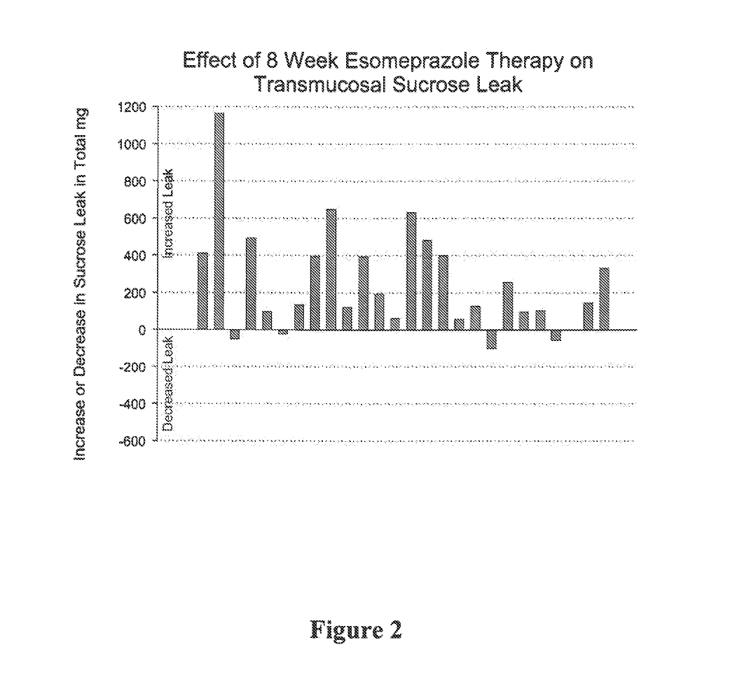

The numerical differences between the result of their second test and the result of their first test are shown for each of the twenty six patients in FIG. 2. Any patient whose initial sucrose permeability test result was greater than 200 mg (suggesting a possible pathology or complication that could skew the results of the test; Meddings et al. (1993) Gastroenter., 104:1619-26) was not reported on here. A total of 36 patients were recruited and completed the test but 11 were removed for this reason. As can be seen, of the remaining 26, 21 (84%) showed increased sucrose leak in their second test at the end of their course of esomeprazole. 14 of 21 in fact showed increased leak above the 200 mg threshold level that others have thought to indicate pathophysiology. The average initial sucrose leak was 72 mg±9 mg (SEM) vs. a mean final sucrose leak of 325 mg±56 mg, an increase of 451%. This difference is statistically significant (P<0.001, Student's t test). If all 37 patients who entered and completed the study are evaluated (without regard to the magnitude of the initial leak, the average initial leak is 179 mg (±39 mg) vs. a final leak of 307 mg (±44 mg), a 171% increase which is also statistically significant (P<0.05). 9 of the 11 patients removed from the study had either medical complications such as hiatal hernia, esophagitis or gastritis or medication use such as ibuprofen or aspirin that could contribute to a higher than normal initial sucrose leak.

In either data set, it is obvious that not all patients exhibited increased leak by the end of the course of esomeprazole. Non compliance, individual differences in terms of reaction to esomeprazole, and the basic study variables described above may all be at play in this observation.

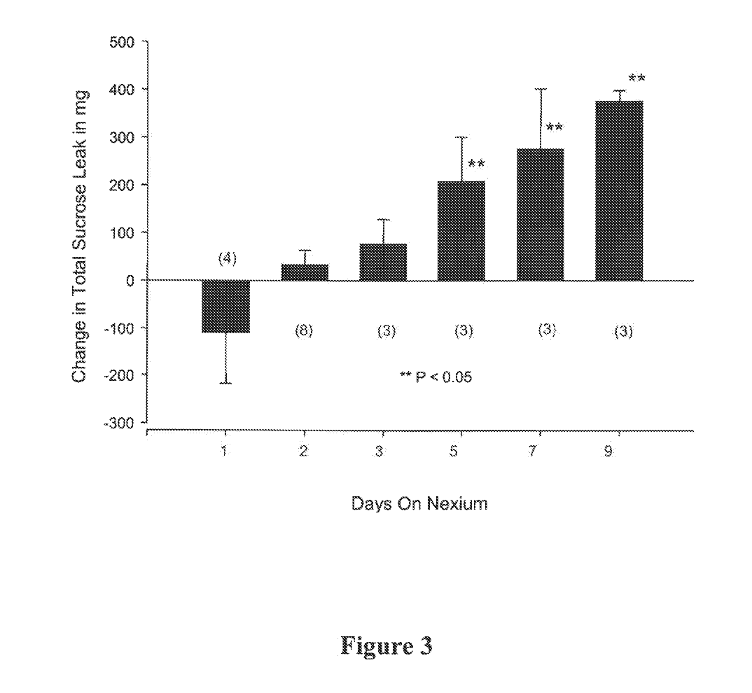

The next study was begun to reduce the (disease) variables that may be at play in working with a GERD population and simultaneously assess the time course with which a PPI leak may establish. As described above, a volunteer group of healthy controls without any active GI disease or prior surgeries was recruited. Volunteers were randomly assigned to duration groups of 1, 2, 3, 5, 7 or 9 days. These subjects performed an initial sucrose leak test then began a 1, 2, 3, 5, 7 or 9 day course of esomeprazole (40 mg, once/day). The night following their last morning dose of esomeprazole, the subject performed their second sucrose permeability test. The numerical difference between the second and the first sucrose permeability test is shown as a function of days on esomeprazole in FIG. 3. As in FIG. 2, a rising bar indicates greater leak in the second test, a falling bar indicates a lower leak at the second test. A statistically significant elevation of leak at the time of the second test was not manifest until the fifth day, interestingly the interval in which plasma levels of the drug are known to increase substantially and plateau (Hassan-Alin, M. (2000) Eur. J. Clin. Pharma., 56:665-70). However the average leak of the second test was greater than the first by the second day and then consistently climbed further out through the nine days. By day 9 in this study, a leak was observed in the second test that on average was almost 400 mg greater than the first (pre esomeprazole) test.

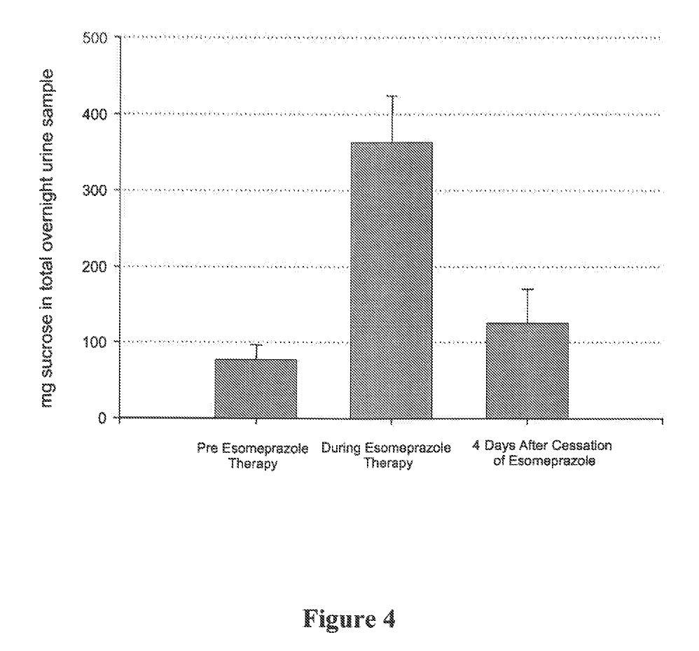

In this study each test subject also performed a third and final sucrose permeability test 4 days after finishing their course of esomeprazole. The results indicate that the leak decreases to near baseline by this time. FIG. 4 shows the average leak values for those who were on esomeprazole for 5, 7 or 9 days (grouped collectively [n=9] since blood levels are known to plateau at the fifth day). The figure gives the average leak of the first (pre esomeprazole) test, the second test and the third (4 day post-esomeprazole) test. Although the second test is statistically different from the first or the third (P<0.01), the third test is not statistically different from the first, evidencing the relative rapid reversibility of the leak phenomenon.

In addition to the above patient-based studies, the effects of omeprazole and esomeprazole in an animal model have been observed. In studies with Sprague Dawley rat gastric corpus, a similarly dramatic PPI-induced leak to nonelectrolytes was observed. Previously, Hopkins et al. (J. Pharm. Pharmacol. (2002) 54:341-7) showed an omeprazole-induced leak to D-mannitol (MW=180) in rat stomach. However, the PPI-induced leak identified by Hopkins et al. only occurred after acid secretion was first stimulated by the administration of an acid secretagogue.

### Example 2

### Induced Leak Admits Large Molecules (e.g., Peptides)

Proton pump inhibitors, the second most prescribed class of drugs in the United States today, are commonly used for the treatment of acid-related disorders including gastric ulcers and reflux esophagitis (Wilde et al. (1994) Drugs 48:91-132). These popular drugs, whose parent compound is omeprazole, function by specifically inhibiting the gastric H⁺/K⁺-ATPase, the pump responsible for gastric acid secretion (Wallmark et al. (1985) Scand. J. Gastroenterol. Suppl., 108:37-51; Larsson et al. (1983) Gastroenterology 85:900-907; Olbe et al. (1986) Scand. J. Gastroenterol. Suppl., 118:105-107). Omeprazole and related proton pump inhibitors (PPIs), like lansoprazole and esomeprazole, effectively suppress basal and stimulated gastric acid secretion and can thereby alleviate symptoms of gastroesophageal reflux disease (GERD) and dyspepsia. PPIs are remarkably well-tolerated drugs and confer little adverse effects in both short-term and long-term therapy. The most commonly reported side effects of omeprazole are diarrhea (in 1-3% of patients), headache (in 0.5-2.4%) and nausea (in 0.9-2%), all of which are generally mild and often short-lived (Wilde et al. (1994) Drugs 48:91-132).

As described herein, clinical studies have shown that patients (with GERD as well as healthy controls) undergoing PPI therapy exhibited increased transmucosal leak of sucrose (a paracellular probe), suggesting the development of a gastric paracellular leak associated with PPI therapy. Here, ex vivo addition of omeprazole to isolated rat gastric corpus mucosa is studied and PPI-induced transepithelial leak of radiolabeled probes (mannitol, sucrose, polyethylene glycol) across the mucosa is examined.

### Methods

### Rat Euthanasia and Tissue Extraction

In each experiment, one adult male Sprague Dawley rat was sacrificed by decapitation. The stomach was removed, flushed, and stripped of its outer serosal membrane, while being frequently moistened with chilled, unbuffered saline. Two pieces of corpus mucosa were mounted in modified Ussing chambers, with exposed tissue surface area of 1.13 cm². Chambers were connected to gas-lift reservoirs, with a fluid volume of 17 mL per hemichamber. The mucosal hemichamber was filled with unbuffered saline (with 17 mM glucose) and kept aerated and perfused with 100% O₂ at 37° C. The serosal hemichamber was filled with bicarbonate buffered saline (with 17 mM glucose) and kept aerated and perfused with 95% O₂/5% CO₂. Water-jacketed reservoirs maintained chamber saline at 37° C.

### Buffers and Ussing Chamber Set-Up

Kreb's Ringer bicarbonate-buffered saline was gassed with 95% O₂/5% CO₂ and was used to bathe the serosal side of the gastric mucosa. KRB contained NaCl (140 mM), KCl (5 mM), CaCl₂-2H₂0 (1.25 mM), MgSO₄ (1.1 mM), Na₂HPO₄ (2.5 mM), Na₂H₂PO₄—H₂0 (0.5 mM), NaHCO₃ (2.5 mM), and glucose (17 mM). KRB was adjusted to a pH of 7.3-7.4. Osmolarity was ˜285 mOsm.

Unbuffered saline was gassed with 100% O₂ and was used to bathe the mucosal side of the gastric mucosa. Unbuffered saline contained NaCl (140 mM), KCl (5 mM), CaCl₂-2H₂O (1.25 mM), MgSO₄ (1.1 mM), and glucose (17 mM) and was also maintained at pH 7.3-7.4 and had an osmolarity of ˜285 mOsm.

High glucose concentrations in the salines in addition to enhanced saline oxygenation (provided by large volume reservoirs) were found essential to sufficiently stimulate acid secretion, presumably because of the high mitochondrial content and aerobic metabolism of parietal cells actively secreting acid.

### Acid Secretion Stimulation

Approximately 30 minutes after tissues were mounted in Ussing chambers, dibutyrl cyclic AMP (dB-cAMP) (Sigma, St. Louis, Mo.) was added to the serosal sides of both chambers to a concentration of 1 mM in order to stimulate acid output by the tissue. In previous studies with isolated rat gastric mucosa in vitro, omeprazole has been found to inhibit both basal acid secretion and acid output stimulated by either histamine or by dB-cAMP (Wallmark et al. (1985) Scand. J. Gastroenterol. Suppl., 108:37-51). Although in a previous study with isolated rat gastric mucosa, histamine adequately stimulated acid secretion in the stomach tissue (Hopkins et al. (2002) J. Pharm. Pharmacol., 54:341-347), in the instant studies histamine was an ineffective secretagogue and dB-cAMP was used in all experiments.

### Measurement of Transepithelial Electrical Characteristics and Mucosal Compartment pH

In order to confirm stimulation of acid secretion by secretagogue addition and to verify the efficacy of the PPI, mucosal fluid compartment pH was recorded at intervals throughout the experiment with a manual electrode (Denver Instruments, Denver, Colo.). Ag/AgCl voltage and current electrodes were used to measure PD (potential difference) and ISC (short circuit current) approximately every five minutes for the duration of the experiment, as previously described (Hameed et al. (2004) Dig. Dis. Sci., 49:1381-1386), using two single-channel current/voltage clamps (McGrath Research and Technology). Ohm's Law was used to calculate transepithelial electrical resistance across the tissue.

Transepithelial Flux Experiments with Radiolabeled Probes

45 minutes following addition of dB-cAMP, radiolabeled probes were added to the serosal fluid compartment of each hemichamber. 12.5 μCi of ¹⁴C-[D]-mannitol (PerkinElmer, Waltham, Mass.), ¹⁴C-sucrose (GE Healthcare, Piscataway, N.J.), or ¹⁴C-polyethylene glycol (Amersham, Piscataway, N.J.) was added to the serosal fluid (17 mL) along with 0.1 mM unlabeled probe molecule to minimize non-specific binding of the isotope. 100 μl samples were taken from the mucosal fluid at fifteen minute intervals for a duration of 105 minutes for liquid scintillation counting. Immediately following the mucosal sampling at 45 minutes flux time, 200 μM omeprazole (in DMSO) was added to the mucosal and serosal hemichambers of the experimental setup and 12.6 μl of DMSO (equivalent experimental volume) was added to the mucosal and serosal fluids bathing the control tissue. Uncharged probes of various molecular weights were used to investigate the size of the PPI-induced transepithelial leak: mannitol (MW 182.2), sucrose (MW 342.3), and polyethylene glycol (MW 4,000).

### Drug Studies

In order to determine if other drugs in the omeprazole class also induce a transmucosal gastric leak, similar experiments were performed with 200 μM lansoprazole (Sigma) and 100 μM esomeprazole (AstraZeneca, London, UK). 100 μM esomeprazole is equivalent to 200 μM omeprazole because of its stereochemical purity. Three experiments were performed with each PPI, examining gastric permeability to ¹⁴C-mannitol.

### Dose-Dependence Studies

To determine the dose-dependence of the omeprazole-induced transepithelial leak, gastric permeability to ¹⁴C-mannitol was assessed as described, using various concentrations of omeprazole (1 μM, 5 μM, 10 μM, 25 μM, 100 μM, and 200 μM). Three experiments were performed at each omeprazole concentration.

### Results

### Omeprazole Stimulates Short Circuit Current and Decreases Transepithelial Resistance

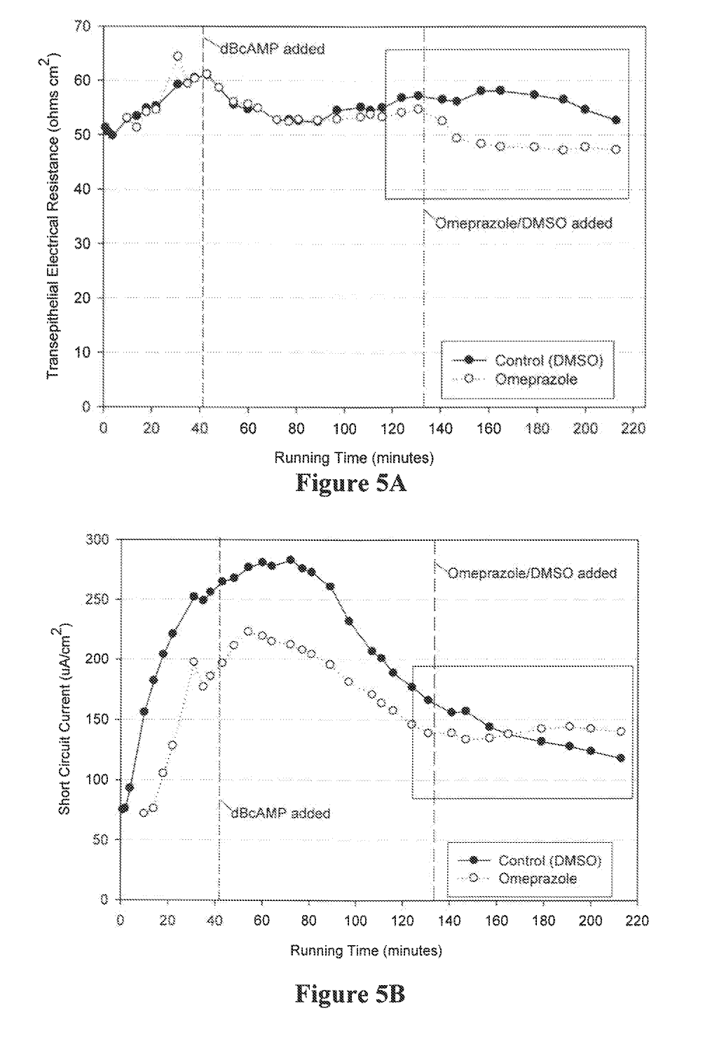

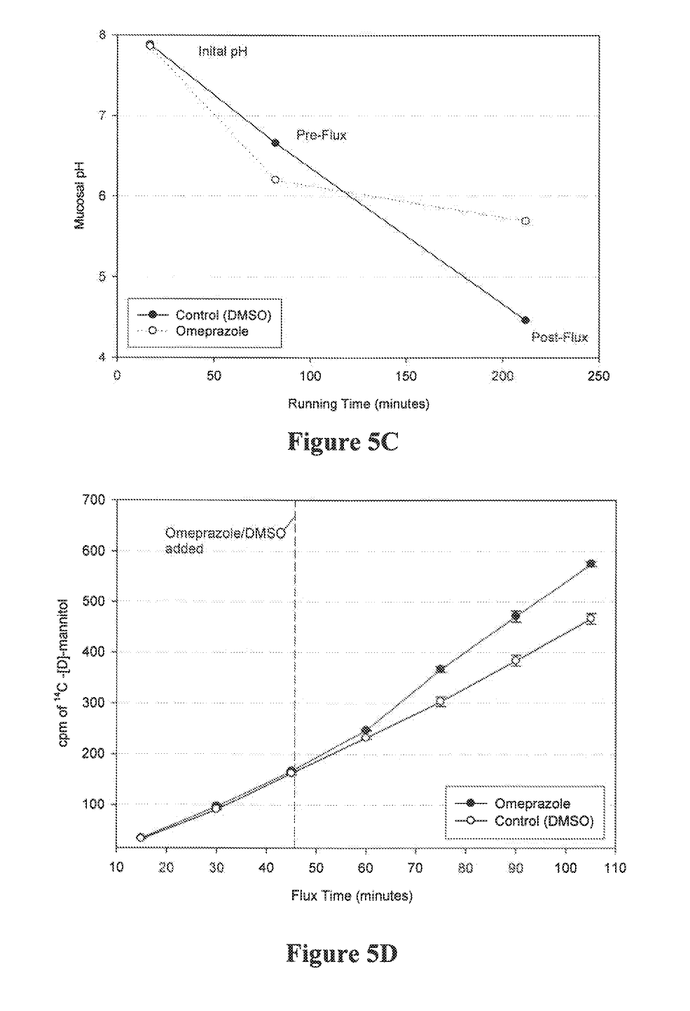

Upon addition of 200 μM omeprazole to both hemichambers, a modest decrease in transepithelial resistance was observed and was accompanied by equally rapid modest stimulation of short circuit current (FIGS. 5A and 5B). The drop in transepithelial resistance occurred immediately upon addition of omeprazole and ranged from 7.6 to 13.7%, over six individual experiments, frequently reversing over the subsequent 60 minutes.

### Omeprazole Inhibits Acid Secretion in a Dose-Dependent Manner

Ex vivo addition of omeprazole, lansoprazole, or esomeprazole to isolated rat gastric corpus in Ussing chambers inhibited acid secretion as exhibited by a decline in the rate of mucosal acidification (FIG. 5C). This was often followed by a gradual increase in mucosal pH. Inhibition of acid secretion by omeprazole was dose-dependent and plateaued at 25 μM omeprazole, where 100% inhibiton of acid secretion (as defined by no further decrease in mucosal fluid pH) was observed.

### Omeprazole Increases Paracellular Permeability of Gastric Mucosa to ¹⁴C-[D]-Mannitol

To follow up on the previous observation of omeprazole-induced decrease of transepithelial resistance, a second indicator of transepithelial leak was also employed. Transepithelial flux of ¹⁴C-[D]-mannitol is a well-described method for observing induced increases in transepithelial paracellular permeability (Hameed et al. (2004) Dig. Dis. Sci., 49:1381-1386; Mullin et al. (1997) J. Cell Physiol., 171:226-233; Mullin et al. (2005) Mol. Biol. Cell, 16:5538-5550). The flux of 0.1 mM ¹⁴C-[D]-mannitol (MW 182.17) across the gastric mucosa increased by a mean of 68.72%+/−8.32 after the addition of 200 μM omeprazole (Table 2 and FIG. 5D). This increase in flux, which was significantly greater than that of the pair-matched, vehicle control (P<0.01), occurred within 15 minutes after addition of the PPI to the chamber and is indicative of a significant increase in, specifically, paracellular transmucosal permeability of the gastric epithelium.

TABLE 2
Transmucosal flux values of ¹⁴C-labeled probes across rat
corpus, before and after treatment with 200 μM Omeprazole.

| | | | | % Increase | Statistical |
|---|---|---|---|---|---|
| Probe | M.W. | Pre-Omeorazole | Post-Omeorazole | in Flux | Significance |
| [D]-Mannitol | 182 | 22.45 +/− 0.61 | 37.81 +/− 1.32 | 68.72 +/− 8.32 | P = 0.01 |
| Sucrose | 342 | 11.71 +/− 3.39 | 24.19 +/− 4.67 | 118.17 +/− 26.26 | P = 0.03 |
| Polyethylene | 4,000 | 5.10 +/− 1.56 | 18.43 +/− 2.60 | 350.46 +/− 148.23 | P = 0.02 |
| glycol | | | | | |

For each probe, three individual experiments (with separate animals) were performed, as described in Methods. All flux values are expressed in ρmoles/min/cm² and represent the mean +/− standard error. Statistical significance was determined by comparing the “pre” and “ post” transepithelial flux rates of radiolabeled probes before and after addition of omeprazole, using a paired Student's t-test. There was no statistically significant difference in flux values of the corresponding, pair-matched, vehicle controls, before and after addition of DMSO.

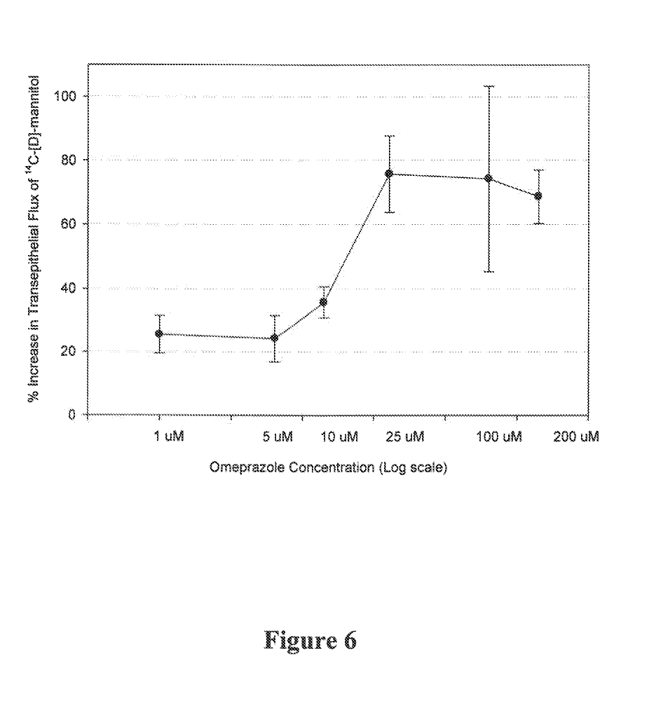

Like omeprazole's dose-dependent inhibition of acid secretion, a similar dose-dependence exists for the permeability increase induced by omeprazole. A marked transepithelial permeability increase in the gastric tissue first occurred at a concentration of 25 μM omeprazole (with no significant effect from 1, 5, or 10 μM) and plateaued thereafter at concentrations of 100 μM and 200 μM (FIG. 6). This inflection point of the permeability effect (25 μM omeprazole) quantitatively corresponds with the point of maximal acid secretion inhibition.

### Omeprazole Increases Paracellular Permeability of Gastric Mucosa to Larger Radiolabeled Probes

Similar PPI-induced permeability effects were observed for the flux of 0.1 mM ¹⁴C-sucrose (MW 342.3) across the gastric mucosa, which increased by an average of 118.17%+/−26.26 upon addition of 200 μM omeprazole (Table 2). This increase was also statistically significant relative to the vehicle control (P<0.03) for the three studies performed. 200 μM omeprazole also induced a gastric leak to ¹⁴C-polyethylene glycol (PEG) (MW 4000). Flux of 0.1 mM ¹⁴C-PEG increased by 350.46%+/−148.23 following omeprazole addition (Table 2), a significantly greater increase than the time-matched vehicle control (P<0.02), for the three studies performed. Thus, 200 μM omeprazole allows for increased transit of probe molecules as large as 4,000 MW.

Lansoprazole and Esomeprazole also Produce Transmucosal Gastric Leak

Lansoprazole and esomeprazole, other proton pump inhibitors of the omeprazole class, also produced a transmucosal gastric leak to ¹⁴C-[D]-mannitol under similar conditions. While 200 μM omeprazole increased flux of ¹⁴C-[D]-mannitol across the gastric corpus by 68.72%+/−8.32, 200 μM lansoprazole increased flux by 83.35%+/−20.71 (Table 3). Esomeprazole, the S-isomer of omeprazole, increased permeability to ¹⁴C-[D]-mannitol by 101.62%+/−10.04, at a concentration of 100 μM (Table 3). Thus, the induced transmucosal leak in the gastric lining is not restricted to omeprazole but is a function of other PPI drugs in the omeprazole class.

TABLE 3
Transmucosal flux values of ¹⁴C-[D]-mannitol across rat corpus,
before and after treatment with various proton pump inhibitors.

| | | | | Statistical |
|---|---|---|---|---|
| PPI | Pre-PPI | Post-PPI | % Increase in Flux | Significance |
| Omeprazole | 22.45 +/− 0.61 | 37.81 +/− 1.32 | 68.72 +/− 8.32 | P = 0.01 |
| (200 μM) | | | | |
| Lansoprazole | 17.47 +/− 1.06 | 31.45 +/− 2.23 | 83.35 +/− 20.71 | P = 0.04 |
| (200 μM) | | | | |
| Esomeprazole | 18.26 +/− 2.11 | 36.39 +/− 2.38 | 101.62 +/− 10.04 | P = 0.0003 |
| (100 μM) | | | | |

For each PPI, three individual experiments (with separate animals) were performed, as described in Methods. All flux values are expressed in ρmoles/min/cm² and represent the mean +/− standard error. Statistical significance was determined by comparing the “pre” and “post” transepithelial flux rates of radiolabeled probes before and after addition of omeprazole, using a paired Student's t-test.

### Discussion

PPI therapy is generally remarkably well-tolerated and, by itself, confers little adverse side effects. Therefore, the PPI induced leak is benign. In both short-term (<12 weeks) and long-term (>10 years) treatment, omeprazole has demonstrated an excellent tolerability profile with rare, and typically harmless, gastrointestinal side effects (Wilde et al. (1994) Drugs 48:91-132). The tolerability profile and adverse effects associated with omeprazole compare favorably to those of histamine H₂-receptor antagonists (Wilde et al. (1994) Drugs 48:91-132; Joelson et al. (1992) Digestion 51:93-101).

Rare side effects associated with omeprazole include skin reactions such as urticaria, angioedema, vasculitic rash, and acute disseminated epidermal necrosis, (Wilde et al. (1994) Drugs 48:91-132; Cox, N. H. (1992) Lancet, 340:857; Haeney, M. R. (1992) BMJ 305:870), haematemesis (Wilde et al. (1994) Drugs 48:91-132), gynaecomastia (Wilde et al. (1994) Drugs 48:91-132; Lindquist et al. (1992) BMJ 305:451-452; Santucci et al. (1991) N. Engl. J. Med., 324:635), impotence (Lindquist et al. (1992) BMJ 305:451-452; Bank et al. (1991) Gastroenterology 100:A29), weight gain (Raoul et al. (1992) Dig. Dis. Sci., 37:1308-1309) and priapism (Dutertre et al. (1991) Lancet 338:1022). Also, there are isolated reports of peripheral neuropathy (Sellapah, S. (1990) Br. J. Gen. Pract., 40:389), acute interstitial nephritis (Christensen et al. (1993) Lancet 341:55; Kuiper, J. J. (1993) Am. J. Med., 95:248; Ruffenach et al. (1992) Amer. J. Med., 93:472-473), lichenoid eruptions (Lee et al. (1989) Med. J. Aust., 150:410), haemolytic anaemia (Marks et al. (1991) Amer. J. Gastr., 86:217-218), peripheral oedema (Brunner et al. (2001) Dig. Dis. Sci., 46:993-996), fulminant hepatic failure (Jochem et al. (1992) Amer. J. Gastr., 87:523-525), hyponatraemia (Gonthier et al. (1993) Presse Med., 22:176), thrombocytopenia (Rudelli et al. (1993) Presse Med., 22:966) and myopathy (Garrote et al. (1992) Lancet 340:672), all associated with PPI therapy. Another obvious causal consequence of PPI therapy is bacterial overgrowth in the upper GI tract resulting from perhaps the most obvious consequence of PPI therapy, namely a much less acidic luminal pH (Fried et al. (1994) Gut 35:23-26).

In addition to the adverse symptomatic effects associated with proton pump inhibitors, a number of morphological and histopathological changes to gastric tissue also may accompany use of these drugs, such as increased gastric epithelial cell proliferation (Kakei et al. (1995) Biochem. Biophys. Res. Comm., 214:861-868), parietal cell hyperplasia and protrusion (Jalving et al. (2006) Aliment Pharmacol. Ther., 1:9), enhanced antral mucosal turnover (Zhang et al. (1998) Dig. Dis. Sci., 43:2764-2770), mucosal thickening (Rindi et al. (2005) Eur. J. Gastroenterol. Hepatol., 17:559-566; Norsett et al. (2005) Physiol. Genomics 22:24-32), reduction in chief cells (Hagiwara et al. (2005) Dig. Dis. Sci., 52:988-994), and increased frequency of fundic polyps (Jalving et al. (2006) Aliment Pharmacol. Ther., 1:9). Typically these morphological changes are accompanied by a decrease in incidence of gastric atrophy and inflammation (Maton et al. (2001) Drug Safety 24:625-635). They may also promote angiogenesis (Sikiric et al. (1999) J. Phys. Paris, 93:479-485).

These various morphological effects of PPIs have been attributed to elevated levels of gastrin and progastrin, resulting from the PPI-induced increase in intragastric pH. Gastrin, a trophic hormone and selective mitogen, stimulates cell division in the stomach's proliferative zone and stimulates mucosal growth and differentiation (Walsh, J. H. (1990) Digestion 47 Suppl 1:11-16, 49-52; Hollande et al. (2001) J. Biol. Chem., 276:40402-40410; Weber et al. (1985) J. Clin. Invest., 75:306-309). The intragastric pH increase resulting from omeprazole therapy stimulates expression of gastrin mRNA and gastrin synthesis and release from the antrum. It also decreases somatostatin mRNA levels (Walsh, J. H. (1990) Digestion 47 Suppl 1:11-16, 49-52). This state of hypergastrinaemia, resulting from PPIs, stimulates proliferation of the oxyntic mucosal stem cells and contributes to mucosal thickening in response to acid inhibition (Walsh, J. H. (1990) Digestion 47 Suppl 1:11-16, 49-52). Prolonged acid inhibition has also been found to increase levels of transforming growth factor-α (Scheiman et al. (1997) Dig. Dis. Sci., 42:333-341). As described below, however, the newly-discovered PPI-induced leak may allow for still another mitogen to play a role in these gastric morphological changes.

It is known that omeprazole and pantoprazole have effects on intracellular calcium signaling, which influences cardiac contractility (Schillinger et al. (2007) Circulation, 116:57; Sarioglu et al. (2000) Life Sci., 66:1411-1421; Naseri et al. (2006) Eur. J. Pharmacol., 531:226-231; Yildirim et al. (2001) Life Sci., 69:435-442). PPIs can also affect other cytoskeletal-mediated process such as neutrophil chemotaxis (Oliveira et al. (2007) Inflamm. Res., 56:105-111), gallbladder relaxation (Aydin et al. (2003) J. Gastroenterol., 38:765-771), smooth muscle activity (Yildirim et al. (2001) Life Sci., 69:435-442), and vas deferens contractility (Yenisehirli et al. (2006) Pharmacol. Res., 54:397-405) by altering intracellular calcium homeostasis (Aydin et al. (2003) J. Gastroenterol., 38:765-771). Similarly, omeprazole and lansoprazole have been found to affect arterial contractions by modulating intracellular calcium (Naseri et al. (2006) Eur. J. Pharmacol., 531:226-231). Lansoprazole has additionally been found to activate mitogen-activated protein kinases (MAPK) (Suzuki et al. (2004) Wound Rep. Regen., 12:93-99), and MAPK is a known regulator of tight junction permeability (Clarke et al. (2000) Adv. Drug Delivery Rev., 41:283-301; Chen et al. (2000) Mol. Biol. Cell, 11:849-862). Such effects on calcium homeostasis and signaling intermediates such as MAPK may influence cytoskeletal elements of GI epithelia and contribute to a PPI-induced tight-junctional leak.

Also, it has been previously reported that lansoprazole and omeprazole, at doses of 10⁻⁴-10⁻⁶ mol/L (clinically relevant doses) inhibit the expression of adhesion molecules such as endothelial cell intercellular adhesion molecule-1 (ICAM-1) and vascular adhesion molecule-1 (VCAM-1), resulting in attenuated adherence between neutrophils and endothelial cells (Yoshida et al. (2000) Alim. Pharma. Therap., 14:74-81). Similarly, changes in epithelial-mesenchymal signaling have been linked to increases in gastrin expression, another effect of PPI therapy (Varro et al. (2007) Amer. J. Phys.-Gastr. Liver Phys., 292:G1133). Thus, without being bound by theory, another possible explanation of the PPI-induced leak may be alterations in cellular adhesion molecules in turn affecting tight junctional proteins.

It is also plausible that PPIs may be producing a leak by inducing cell death. PPIs have been found to induce apoptosis in human B-cell tumors by production of reactive oxygen species (De Milito et al. (2007) Cancer Res., 67:5408). Additionally, omeprazole is known to induce apoptosis in Jurkat cells (Scaringi et al. (2004) Int. J. Immunopathol. Pharmacol., 17:331-342). Pantoprazole can selectively induce apoptosis in gastric cancer cells (Yeo et al. (2004) Clin. Cancer Res., 10:8687), while other ATPase inhibitors induce apoptosis in leukemia cells (Shiono et al. (2002) Anticancer Res., 22:2907-2911). Also, acid inhibition has been linked to an increased expression of genes associated with apoptosis and increased inflammatory and stress responses (Rindi et al. (2005) Eur. J. Gastroenterol. Hepatol., 17:559-566; Norsett et al. (2005) Physiol. Genomics 22:24-32). On the other hand, omeprazole has been demonstrated to have an antiapoptotic role and possess antioxidant characteristics in rat gastric tissue (Biswas et al. (2003) J. Biol. Chem., 278:10993-11001). In yet another study, omeprazole and esomeprazole were found to have no effect on apoptosis, p53 expression, EGFR expression, or proliferation of gastric epithelial cells (Hritz et al. (2005) World J. Gastroenterol., 11:4721-4726). Thus, it is possible that the PPI-induced transepithelial leak may result from cell death. However, the very rapid time course of PPI-induced leak shown herein and the leak specificity shown herein argue against cell death-mediated leak.

Despite all of the above, it important to note that PPI therapy is remarkably well-tolerated and, by itself, confers little adverse side effects. In both short-term (<12 weeks) and long-term (>10 years) treatment, omeprazole has demonstrated an excellent tolerability profile with rare, and typically harmless, gastrointestinal side effects.

Regardless of its cellular mechanism, the PPI-induced transepithelial leak allows for oral delivery of medications which must currently be injected or I.V. infused, as such drugs could avoid metabolic degradation in the intestine if they diffuse into the bloodstream across the upper portion of the GI tract. The GI leak described herein may explain, for example, why eradication of Helicobacter pylori is more effective when amoxicillin is coupled with omeprazole than by amoxicillin alone (Bell et al. (1993) Scand. J. Gastroenterol. Suppl. 196:7-11; Labenz et al. (1993) Am. J. Gastroenterol., 88:491-495; Unge et al. (1989) Scand. J. Gastroenterol. Suppl., 167:49-54). PPIs might facilitate the diffusion of antibiotics from the serosal fluid to the gastric lumen, by a gastric mucosal permeability change, allowing for increased efficacy in treatment.

In addition to the drug delivery aspects of the PPI-induced leak, the risks of leakage of secondary small-molecule medications which a patient may be taking in combination with PPIs may be assessed. Digoxin toxicity has recently been reported to be associated with omeprazole therapy (Kiley et al. (2007) South Med. J., 100:400-402). Omeprazole increased serum levels of digoxin by more than 350%, “possibly due to increased absorption in the stomach”, since these two drugs would not likely have a pharmacokinetic interaction (Kiley et al. (2007) South Med. J., 100:400-402). Because PPIs are so widely used, and often assumed to be inoccuous, the effects of the PPI-induced leak on other oral medications that a patient has been prescribed may be studied and/or monitored. The inhibitory effects of PPIs on liver cytochrome P450s and consequent lengthening of the half life of certain drugs in the bloodstream do not fully explain the ability of PPIs to increase the blood levels of certain drugs. The PPI-induced leak pathway for these drugs to enter the bloodstream significantly increases their blood levels.

This study established the increased permeability of rat gastric mucosa to non-electrolyte probes of various sizes in tissue treated with PPIs, particularly different drugs of the omeprazole class.

### Example 3

### PPI Induced Leak Allows Only Certain Small Molecules to Pass

Two clinically relevant, small molecule drugs, digoxin (780 MW) and phenytoin (252 MW), with narrow therapeutic windows and whose blood levels must therefore be carefully titrated, were studied. Digoxin is used to treat congestive heart failure and supraventricular arrhythmias (Mulrow et al. (1984) Ann. Intern. Med., 101:113-7) and phenytoin is an anticonvulsant medication (Appleton et al. (2008) Cochrane Database System. Rev., 3:CD001905). Both molecules are below the size limit (10 kDa) of the omeprazole-induced leak.

The instant study is on the effect of the PPI-induced leak on blood levels of these drugs. The ability of a PPI-induced gastric leak to allow these drugs to cross the gastric barrier and thus allow for a significant increase in net uptake of these drugs into the bloodstream was studied. The gastric leak produced by PPI provides drugs with an additional avenue of uptake proximal to the small intestine and not subject to cellular metabolism.

It is already well known that PPIs can cause an elevation in blood levels of certain drugs, such as digoxin, by inhibiting their removal from the bloodstream by liver cytochromes (Peterson, K. U. (1995) Aliment Pharmacol. Ther., 9:1-9). Omeprazole also increases the bioavailability of oral digoxin by suppressing gastric acid production (Robinson et al. (2003) Drugs, 63:2739-54). As demonstrated herein, PPIs can also increase drug uptake into the bloodstream by inducing a transmucosal gastric leak. Notably, the uncontrolled combination of the PPI and the drug may synergize in certain individuals to create dangerous elevations in the blood levels of these drugs. Primary care physicians, as well as gastroenterologists, need to be aware of this potential, unusual drug interaction.

### Materials and Methods

### Animals, Tissue Dissection, Ussing Chambers, Incubation Salines

Sprague Dawley male rats weighing approximately 400 grams were sacrificed by decapitation and the stomach was quickly removed, cut along the greater curvature, and flushed of luminal contents. The serosal membrane was then stripped from the corpus region and two equal sized pieces of corpus mucosa were mounted in two separate Ussing chambers with exposed tissue areas of 1.13 cm². Chambers were connected to 15 mL gas-lift reservoirs filled with un-buffered saline aerated with 100% O₂ and bicarbonate-buffered saline aerated with 95% O₂/5% CO₂ on the mucosal and serosal sides, respectively. Saline temperature was held constant at 37° C. by a jacketed water bath surrounding the reservoirs. 30 minutes at 37° C. was allowed for tissue equilibration. In paired experiments, one tissue served as (vehicle) ‘control’, and the other as omeprazole-treated ‘experimental’.

### Electrophysiology and Mucosal pH

Electrical parameters were monitored throughout the duration of the experiment. Tissues were maintained under open circuit conditions. Ag/AgCl electrodes were bridged to saline in chambers with 3% agarose bridges stored in 1M NaCl. Tissue voltage (potential difference), short circuit current, and transepithelial resistance were recorded approximately every five minutes for the duration of the experiment, by passing a one-second current pulse equal to biological current to bring the potential difference to zero using a current/voltage clamp (McGrath Research & Technology). Transepithelial resistance was calculated by dividing open circuit potential difference by short circuit current (Ohm's law).

Mucosal and serosal saline pH measurements were taken at approximately five minute intervals using a manual electrode (Denver Instruments) to ensure that the tissue was secreting acid. Mucosal pH readings were also taken before and after the addition of omeprazole to document the action of omeprazole on inhibiting acid secretion. Tissue viability was confirmed at the end of each experiment by tissue exposure to 10 mM amiloride in the mucosal fluid compartment in order to inhibit short circuit current.

### Transepithelial Drug Flux Studies

After 30 minutes of tissue equilibration time in Ussing chambers, dibutyrl cyclic AMP (dBcAMP) (Sigma) was added to a final concentration of 1 mM to the serosal fluid chambers to stimulate acid secretion. 45 minutes following dBcAMP addition, radiolabeled drugs under study (3H-digoxin, ¹⁴C-phenytoin) along with their unlabeled forms were added to the mucosal fluid of both chambers to achieve final concentrations of 0.1 mM phenytoin or 0.05 mM digoxin in the mucosal fluid. The addition of the isotope marked the beginning of the flux period. 150 μL or 250 μL samples were taken from the serosal fluid compartment (opposite from which the isotope was added) for liquid scintillation counting (LSC). Samples were initially taken at 30 minute intervals for 90 minutes for the ¹⁴C-phenytoin flux or at 15 minute intervals for 75 minutes for the ³H-digoxin flux to establish the basal permeability rate (ρmoles/min/cm²) across the tissue. 200 μM omeprazole (Sigma) (in DMSO) was then added to the mucosal and serosal sides of one chamber and an equivalent amount of DMSO (Sigma) was also added to both sides of the control chamber. Another series of serosal fluid samples were taken for LSC at 15 or 30 minute intervals for the remaining 60 minutes of ³H-digoxin or ¹⁴C-phenytoin flux experiments, respectfully, to determine flux rate of the radiolabeled drugs after omeprazole addition. In all cases of fluid sampling for liquid scintillation counting, an equal amount of fresh buffered saline was added back to the serosal side to prevent hydrostatic gradients from developing across the tissue. The flux of each isotope (ρmole/min/cm²) was determined by calculating the linear regression slope of the graph of cpm versus time, and converting cpm to picomoles after determining the specific activity of the drug in the mucosal fluid.

### Thin-Layer Chromatography

Additional saline samples were taken from the mucosal and serosal fluid compartments one minute before the addition of omeprazole/DMSO and at the end of the entire flux period to allow for thin-layer chromatography (TLC) analysis of radioactivity that crossed the tissue. TLC was performed to account for the potential contribution of radiolabeled metabolites to total radioactivity crossing the epithelial barrier, in the event of drugs entering gastric epithelial cells and metabolites being effluxed. Saline samples were taken from both surfaces of the tissue to determine possible degradation of the molecule upon exposure to the mucosal surface of the tissue as well as to analyze the chemical nature of the radioactivity that crossed the tissue. Saline samples from digoxin and phenytoin flux experiments were concentrated by evaporation (Savant speed vac) to dryness and then resuspended in small volumes of methanol and 100% ethanol, respectively. TLC was performed on silica gel 60 plates (Kodak) by spotting very small amounts of the concentrated sample. Plates were then placed in a sandwich tank and a variety of mobile phases were employed, including butanol/acetic acid/water (120:30:50), isopropanol/water (120:30), and ethyl acetate/methanol/water (81:11:8). After completion of the mobile phase, plates were sprayed with Kedde reagent to visually detect the location of the digoxin or examined under a 254 UV lamp for detection of phenytoin.

### Results

As stated hereinabove, exposure of gastric mucosa to omeprazole (200 μM) inhibited acid secretion and stopped the decline of mucosal fluid pH. Omeprazole also caused a small, but significant, decrease in transepithelial electrical resistance and a dramatic increase in transepithelial D-mannitol leak.

In the course of the permeability studies it was observed that neither digoxin nor phenytoin, at the concentrations used in these flux studies, had an effect on transepithelial electrical resistance or short circuit current of the gastric mucosa tissue. They were thus not themselves exerting an effect on epithelial barrier properties.

To observe if the omeprazole-induced leak allows for permeation of certain small molecule drugs, ³H-digoxin was added to the mucosal fluid compartment and saline samples were taken at 15 minute intervals from the serosal fluid compartments for a 135-minute flux period. Omeprazole and/or DMSO were added to their respective tissues 75 minutes after the addition of the isotope. Radioactivity present in the serosal fluid compartment was measured as a function of time before and after the addition of omeprazole or DMSO.

When a plot of total serosal radioactivity (cpm) versus time was constructed, a significant difference (in slopes of cpm vs. time) for the omeprazole versus DMSO (vehicle control) conditions was observed. The increase in flux of total radioactivity after omeprazole addition was 84%±7% (SEM) versus an increase of 50%±6% for vehicle control, and these differences were significant (P<0.03, paired Student's t test). Omeprazole therefore caused an increase in the total radioactivity (tritium) crossing the gastric mucosa. This was like earlier results using radiolabeled mannitol, sucrose, and polyethylene glycol as probes, where omeprazole caused dramatic increases in rate of transepithelial diffusion of these molecules. However, mannitol, sucrose, and polyethylene glycol are not only non-metabolizable by gastric tissue, but have negligible uptake into cells. Thus, for these probes, 100% of the cpm appearing in the serosal fluid were the (undegraded) molecule in question. This was not the case for ³H-digoxin, which exhibited evidence of significant metabolism by the gastric tissue.

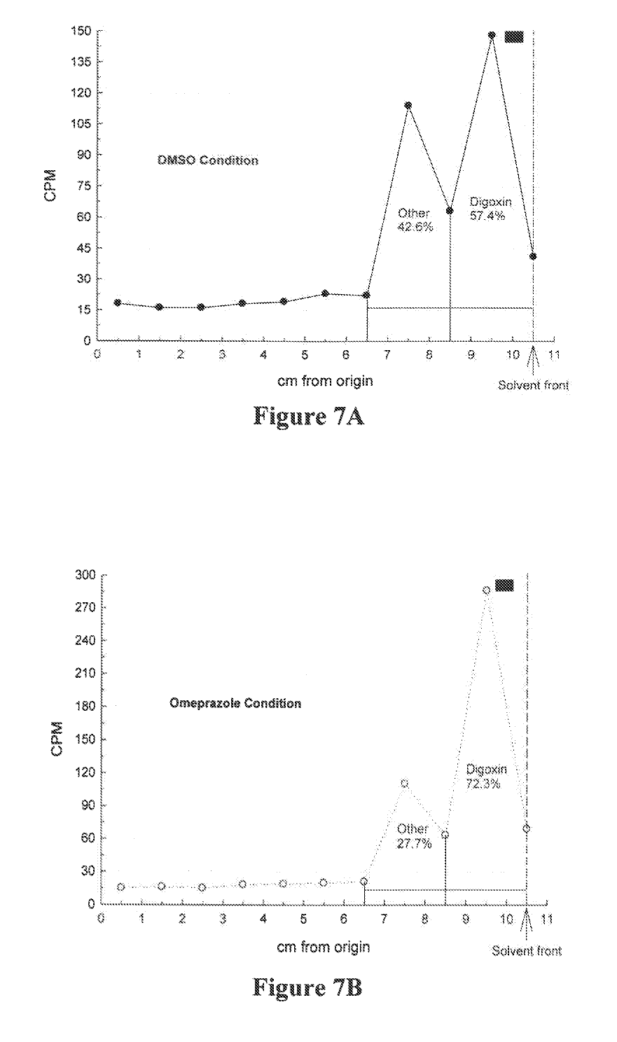

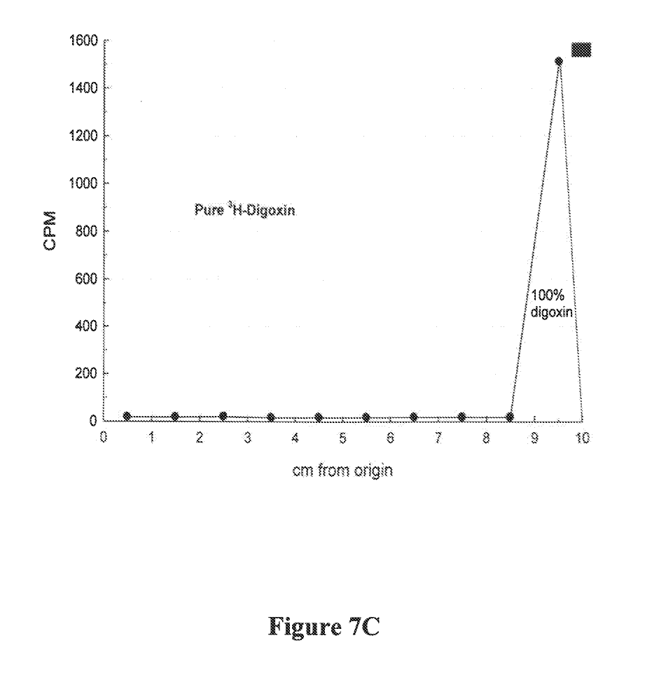

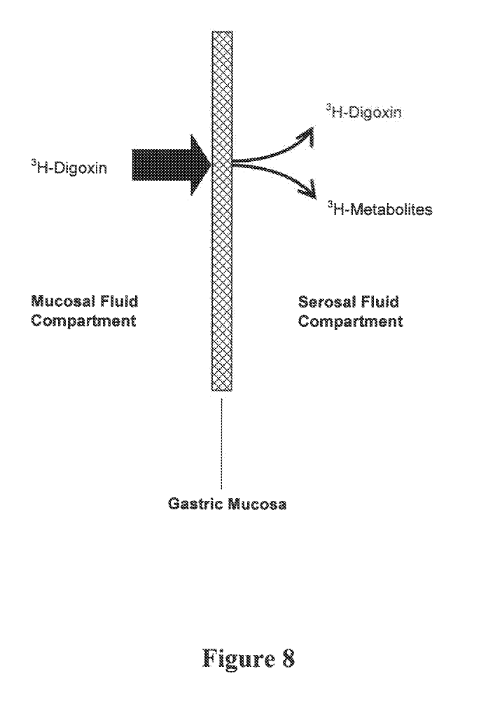

Thin-layer chromatography analysis of total tritium crossing the gastric mucosa was performed on the serosal fluid samples from each ³H-digoxin flux experiment. This analysis showed that not all of the radioactivity coming across the rat gastric corpus mucosa was ³H-digoxin (FIGS. 7A and 7B). The first (and smaller) radiochromatogram peak is likely indicative of a digoxin metabolite, occurring as a result of ³H-metabolite efflux from the cells (FIG. 8). The majority of radioactivity crossing the cell sheet and present in the serosal fluid (typically 54.9%-75.5%) did, however, co-migrate with a known pure digoxin standard (FIG. 7C) as assessed by butanol/acetic acid/water (120:30:50), isopropanol/water (120:30), and ethyl acetate/methanol/water (81:11:8) mobile phases (only the isopropanol/water mobile phase results are shown). This percent of radioactivity co-migrating with pure digoxin was used to adjust net transepithelial fluxes of total tritium, thus eliminating the non-digoxin radioactivity from flux analysis and delivering an adjusted flux of pure digoxin. Following these calculations, the flux rate of digoxin per se (and not simply total radioactivity) also increased significantly after addition of omeprazole (82.2%±7.7% SEM) compared to the paired DMSO vehicle control (50.9%±7.0%) (Table 4).

TABLE 4
Precent increase in flux of radioactive probes crossing rat gastric
corpus mucosa over time after treatment with 200 μM omeprazole.

| | Pre-DMSO | Post-DMSO | % Increase in |
|---|---|---|---|
| | Flux Value | Flux Value* | Flux After |
| Probe | (ρmole/min/cm²) | (ρmole/min/cm²) | DMSO |
| ¹⁴C-Mannitol | 22.7 ± 3.3 | 27.2 ± 2.4 | 23.0% ± 15.3% |
| (0.1 mM) | | | |
| ³H-Digoxin | 4.8 ± 1.0 | 7.2 ± 1.5 | 50.9% ± 7.0% |
| (0.05 mM) | | | |
| ¹⁴C-Phenytoin | 71.9 ± 9.8 | 120.0 ± 14.7 | 69.3% ± 13.9% |
| (0.1 mM) | | | |

| | | | | Statistical |
|---|---|---|---|---|
| | Pre- | Post- | | Significance of % |
| | Omeprazole | Omeprazole | % Increase | Increase Relative to |
| | Flux Value | Flux Value* | in Flux After | Paired Vehicle |
| Probe | (ρmole/min/cm²) | (ρmole/min/cm²) | Omeprazole | Control (DMSO) |
| ¹⁴C-Mannitol | 21.2 ± 5.3 | 31.6 ± 3.9 | 68.7% ± 8.4% | P < 0.05 |
| (0.1 mM) | | | | (n = 3) |
| ³H-Digoxin | 5.1 ± 0.2 | 9.4 ± 0.6 | 82.2% ± 7.7% | P < 0.03 |
| (0.05 mM) | | | | (n = 4) |
| ¹⁴C-Phenytoin | 78.3 ± 11.4 | 119.4 ± 12.9 | 54.4% ± 7.7% | P = 0.35 (N.S.) |
| (0.1 mM) | | | | (n = 3) |

Pre- and post-omeprazole and pre- and post-DMSO (vehicle control) flux values are listed in picomoles/min/cm² ± the standard error of the mean. The post-omeprazole and post-DMSO flux values are corrected values based on thin-layer chromatography results.
The flux values of phenytoin are markedly higher than those of mannitol, suggesting possible transcelluar transit of phenytoin.
Values for digoxin are representative of 4 individual experiments.
Values for phenytoin and mannitol represent 3 experiments.
Statistical analyses were performed using a Student's t-test.
Statistical significance was found between post-DMSO vs. post-omeprazole values of radiolabeled digoxin and mannitol, indicating the omeprazole-induced gastric leak allows these molecules to cross paracellularly at an accelerated rate, as compared to the control.
All values shown represent the mean percent increase in flux for each radiolabeled probe after the addition of omeprazole (or DMSO), ± the standard error of the mean. Percentages were calculated by subtracting the pre-omeprazole or pre-DMSO ρmoles/min/cm² flux values from the post-omeprazole or post-DMSO ρmoles/min/cm² flux values and then dividing that value by the pre-omeprazole or pre-DMSO value. The increased flux values in the presence of DMSO may represent a DMSO effect and/or gradual and minor cell death in the preparations over time.
Overall viability of the preparations was attested to by amiloride-sensitive short circuit current and potential difference out to the end of the experiments.
The percentage of DMSO in the incubation saline never exceeded 0.5%.

Thin-layer chromatography analysis also showed proportionally more radiolabeled digoxin (72.3% vs. 57.4%) and proportionally less digoxin metabolites (27.7% vs. 42.6%) present in the serosal fluid when omeprazole has induced a transepithelial leak (FIG. 7, panel B vs. panel A). This underscores the fact that the omeprazole-induced leak allows for digoxin permeation across the gastric mucosa. This observation was consistent over 4 experiments. Although this low n value did not allow for statistical significance, the data was close to the 5% level, with a P value of approximately 0.10 in both Student's t-test and Tukey-Duckworth. This, plus the non-overlap of means+/−standard error, indicates that omeprazole-treatment exhibits proportionally greater transepithelial flux of pure ³H-digoxin as opposed to radiolabeled digoxin metabolites (Table 5).

TABLE 5
Percentage of ³H-digoxin in the serosal fluid
compartment in each condition (omeprazole and
DMSO) after thin-layer chromatography analysis.

| | % Digoxin in Serosal | % Digoxin in Serosal |
|---|---|---|
| Digoxin Flux | Fluid in DMSO (vehicle | Fluid in Omeprazole |
| Experiment | control) Condition | Condition |
| 1 | 76.4% | 83.7% |
| 2 | 59.0% | 72.3% |
| 3 | 57.4% | 73.0% |
| 4 | 27.0% | 72.9% |
| Average | 55.0% ± 10.2% | 75.5% ± 2.7% |

Note the larger amount of digoxin in the omeprazole condition relative to the paired DMSO control. The average ³H-digoxin is listed ± standard error. The percentage of ³H-digoxin in mucosal fluid was never less than 99%. Non-³H-digoxin radioactivity in the serosal fluid compartment is likely digoxin metabolites.

The flux increase observed in the presence of DMSO (vehicle control) is likely due to a characteristic slow, progressive decrease of tissue viability in Ussing chambers over time. This decrease, however, is minor as amiloride-inhibitable short circuit current and potential difference persist throughout the experiments. The background (basal) rate of digoxin flux across the (control) tissue is likely due to edge damage of tissue in the Ussing chamber, a well described issue in these types of studies (Dobson et al. (1968) Am. J. Physiol., 214:719-724; Walser, M. (1970) Am. J. Physiol., 219:252-255). The combined effect of a leak component from tissue deterioration and a leak component from edge damage will detract from (and understate) the perceived magnitude of the flux increase achieved as a result of omeprazole exposure (Svelto et al. (1975) Arch. Intl. Physiologie Biochim., 83:837-843).

Unlike digoxin, ¹⁴C-phenytoin did not come across the gastric corpus mucosa at an accelerated rate in the presence of omeprazole, with a non-significant flux rate increase (54.4%±7.7% SEM) after omeprazole treatment relative to the paired vehicle control (69.3%±13.9%) (Table 4). This was true even after analysis of total ¹⁴C-radioactivity by TLC. In fact, thin-layer chromatography analysis of ¹⁴C in serosal fluid indicated no metabolic breakdown of phenytoin associated with its crossing the gastric mucosa, a result consistent with diffusion through the edge-damage pathway and/or negligible cellular metabolism of phenytoin passing transcellularly. The single peak of radioactivity observed after running radiochromatograms in a variety of solvent systems (butanol/acetic acid/water (120:30:50), isopropanol/water (120:30), and ethyl acetate/methanol/water (81:11:8)) showed 100% co-migration with an established pure phenytoin standard. This indicates all cpm crossing the gastric mucosa were ¹⁴C-phenytoin. However, an omeprazole-induced flux increase for phenytoin did not occur.

### Discussion

In further characterizing the PPI-induced paracellular leak in gastric corpus mucosa, it was observed that this leak allows the small molecule drug, digoxin, to permeate, but not phenytoin. The data clearly indicate digoxin manifesting a transepithelial flux increase in the presence of omeprazole. Phenytoin did not manifest a flux increase under the same conditions. These findings build on the results presented herein showing that the PPI-induced gastric leak occurs in the corpus region of the stomach, allows only molecules of about 4-10 kDa or smaller to permeate, is bidirectional, and depends on luminal acidification.

Phenytoin (252 MW) is not only smaller than digoxin (780 MW), but based on the above findings, is well below the size limit (˜4 to 10 kDa) of molecules able to pass through this leak pathway. It appears counterintuitive, therefore, that the larger molecule (digoxin) crossed the gastric mucosa at an accelerated rate in the presence of omeprazole, while the smaller one (phenytoin) did not.

The flux increase for both drugs observed in DMSO-treated (vehicle control) tissue is likely due to decreased tissue viability over time and edge damage incurred during mounting in Ussing chambers. The resulting basal leak quantitatively detracts from the perceived increased leak seen in the omeprazole-treated tissue. Considering that no corresponding basal leak is likely present in vivo, the relative magnitude of the omeprazole-induced leak would therefore be more pronounced in vivo than reported here in vitro.

In comparing the actual flux values (in ρmoles/min/cm²) of phenytoin versus the inert carbohydrate, mannitol, the absolute amount of phenytoin crossing the gastric mucosa after treatment with omeprazole or DMSO was much higher than the amount of mannitol crossing under the same conditions (Table 5). This indicates phenytoin may be transiting the mucosa transcellularly as well as paracellularly. This is consistent with phenytoin being considerably more hydrophobic than mannitol. Thin-layer chromatography (TLC) results described herein indicate that phenytoin is not being detectably metabolized by gastric epithelia during this transit.

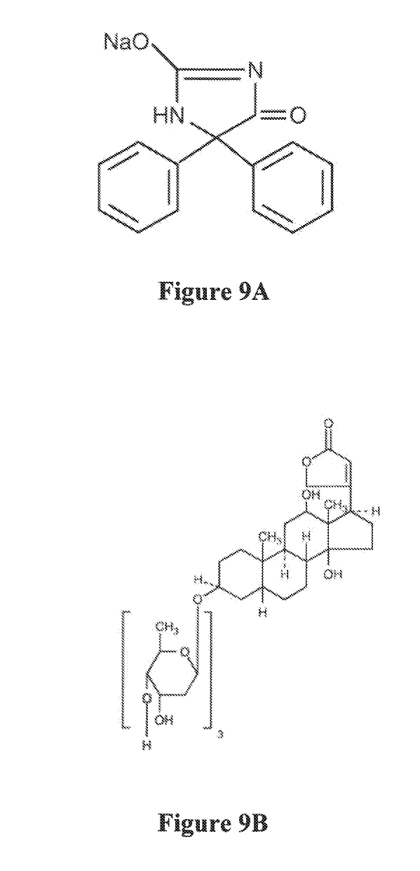

Although phenytoin is a markedly smaller molecule than digoxin, other obvious differences between the two are molecular structure and hydrophobicity (FIGS. 9A and 9B). The extensive number of hydroxyl groups on the digoxin molecule makes it more water-soluble and more capable of hydrogen bond formation than phenytoin. Phenytoin would form much weaker hydrogen bonds and is less hydrophilic than digoxin. Both properties may impact a molecule's ability to diffuse through a tight junction leak. Other molecules shown to pass through the PPI-induced gastric mucosal leak (mannitol, sucrose, and polyethylene glycol) are all capable of extensive hydrogen-bond formation with water molecules and with amino acid residues lining a pore. In the case of transport-mediated hexose uptake into a cell, free hydroxyls and hydrogen bonding-capability are essential for a hexose to pass through the SGLT transporter (Kleinzeller et al. (1980) Biochim. Biophys. Acta, 600:513-29). The hydrophobicity and relative lack of hydrogen bond formation by phenytoin is a possible explanation as to why it cannot pass through a PPI-induced tight junctional pore. This indicates a surprising and unexpected degree of specificity for what is most likely a paracellular leak established by PPI action.

Specific PPIs can interact with the cytochrome P-450 system in the liver causing inhibition of hepatic breakdown of certain drugs, with resultant prolonged elimination from the bloodstream and increased drug half-life (Peterson, K. U. (1995) Aliment Pharmacol. Ther., 9:1-9). In the case of digoxin, PPIs not only interfere with cytochrome-mediated digoxin removal from the bloodstream (Humphries et al. (1999) Aliment Pharmacol. Ther., 13 Suppl 3:18-26) but also open up the gastric barrier for digoxin to enter the blood “upstream” of (and in addition to) its normal uptake site in the intestine. The two separate effects would combine to elevate blood levels of digoxin. In comparison, many reports indicate that PPIs have no effect on the blood levels of phenytoin (Andersson et al. (1990) Ther. Drug Monit., 12:329-33; Bachmann et al. (1993) Br. J. Clin. Pharmacol., 36:380-2; Middle et al. (1996) Clin. Pharmacol. Ther., 34:S72-5; Karol et al. (1999) J. Clin. Pharmacol., 39:1283-9), despite the fact that PPIs are known to interact with cytochromes responsible for phenytoin breakdown (Howden, C. W. (1991) Clin. Pharmakinet., 20:38-49; Andersson, T. (1991) Clin. Pharmakinet., 21:195-212; Cederberg et al. (1989) Scand. J. Gastroenterol., 166:33-42). Interestingly, PPIs were not observed allowing phenytoin to cross the gastric barrier.

Although the instant data shows digoxin diffusing through the omeprazole-induced leak in gastric mucosa, there is still the issue of whether digoxin can diffuse across in clinically significant amounts. Blood levels of digoxin are maintained in a relatively narrow therapeutic range (1.0-2.5 nanomolar; 0.5-2.0 ng/mL). After an oral dose of digoxin (typically 125 micrograms), it was determined whether the amount of digoxin passing through a PPI-induced leak in gastric mucosa would materially increase the final blood concentration of digoxin in a clinical setting.

Considering only the amount of digoxin observed crossing the mucosa without metabolic degradation, the final flux of true ³H-digoxin was approximately 9 picomoles/min/cm² in the presence of omeprazole. A portion of this flux is due to damage of the gastric tissue preparation during mounting in the Ussing chambers. To correct for flux due to edge damage, the flux rate was subtracted prior to omeprazole addition from the flux rate after omeprazole addition. However, the added issue of decline in cell viability over the course of any experiment, and its contribution to overall leak, still remains (this is generally observed in the modest decline in transepithelial electrical resistance over experimental running time). If the flux of ³H-digoxin in the presence of DMSO (vehicle control) is subtracted from the flux of ³H-digoxin in the presence of omeprazole (paired experiments), any possible effects of the omeprazole solvent (DMSO) on barrier function is controlled for, as well as the variables of edge damage and any effect of decline in tissue viability over the 135 minute flux period. Performing these calculations gives us a final omeprazole-dependent ³H-digoxin flux of 1.8±0.4 (SEM) picomoles/min/cm² as seen over 4 paired experiments.

Assuming the surface area of an adult human gastric corpus (the gastric region affected by omeprazole) is 500 cm² (Robertson, D. S. (2005) Med. Hypoth., 64:1127-1131), the flux rate of 1.8 picomoles/min/cm² (calculated above) translates to a total gastric corpus-wide flux of 900 picomoles/minute across a typical human adult stomach. After an oral dose of digoxin (assuming the average human gastric emptying time is 60 minutes), 54,000 picomoles of digoxin could efflux across the gastric mucosa into the bloodstream. This value (54,000 ρmole), however, is based on the 50 micromolar concentration of digoxin in the Ussing chamber mucosal fluid compartment. A typical clinical oral dose of digoxin is 125 micrograms (0.16 micromoles). If 0.16 micromoles of digoxin is dissolved in 100 cc of gastric luminal fluid contents (typical volume of human fasted stomach), the mucosal digoxin concentration equals 1.6 micromolar versus the 50 micromolar used in the above experiments. Since diffusion of digoxin along the paracellular route follows the First Law of Diffusion (flux is proportional to concentration), the total in vivo flux of digoxin is not 54,000 picomoles, but 54,000×(1.6/50.0), or 1728 picomoles. If 1728 picomoles of digoxin enter the typical adult plasma volume of 2.5 liters, then the contribution of the gastric leak to the final plasma concentration of digoxin would be an increase of 0.7 nanomolar. This increase would be medically significant given that the customary desired digoxin serum concentration range is 1.0 to 2.5 nanomolar. Therefore, uptake of digoxin into the bloodstream through a PPI-induced gastric leak significantly elevates serum digoxin levels. The magnitude of flux through this gastric leak pathway is, therefore, clinically relevant. The importance of this finding is further emphasized by a recent study suggesting an even narrower recommended therapeutic window for digoxin (0.5-0.8 ng/mL) because higher serum digoxin concentrations were associated with increased mortality (Rathore et al. (2003) JAMA 289:871-878).

Add the above consideration to the fact that PPIs can inhibit liver cytochromes capable of degrading digoxin (CYP3A4) (Steinijans et al. (1994) Int. J. Clin. Pharmacol. Ther., 32:385-99), as well as increase the bioavailability of oral digoxin by suppressing acid production in the stomach (Robinson et al. (2003) Drugs, 63:2739-54), and the overall conclusion is that several different actions of PPIs can synergize to elevate digoxin levels in the blood. Although not all studies maintain that PPIs do in fact elevate blood digoxin levels (Oosterhuis et al. (1991) Br. J. Clin. Pharmacol., 32:569-72), this is a possibly dangerous clinical situation considering the necessary close titration of digoxin blood levels. At the start of digoxin therapy, a physician carefully titrates and monitors digoxin blood levels until optimal balance is reached. Therefore, the clinical danger resides in a patient currently on drug x (e.g. digoxin), who develops heartburn at some point in the future while still on “drug x”, and then begins PPI therapy. In this scenario, after digoxin blood levels were already carefully titrated and the patient starts taking a PPI, the gastric leak induced by the PPI (and perhaps the cytochrome inhibition) could spike the blood concentration of digoxin to clinically dangerous levels, as has recently been observed clinically (Kiley et al. (2007) South Med. J., 100:400-2).

### Example 4

### Similar Gastric Leak Produced by H2 Blocker

H-2 blockers such as PEPCID® (famotidine) also cause a GI leak. The instant study was first performed with 20 subjects, all healthy controls. Randomly 10 were put on PPIs (20 mg of omeprazole), 10 on H-2 blockers (40 mg of famotidine). These amounts are standard for acid suppression therapy. All 20 performed a baseline sucrose leak test before being given any medication. They were given doses of these drugs in the morning for five days. A sucrose leak test was done the night of the fifth and last day.

The PPI group showed pre-med leak results of 42 mg+/−11 mg (SEM) (n=10) vs. a post med leak of 212 mg+/−39 mg. Paired t test has a P=0.003.

The H-2 group showed a pre-med leak of 50 mg+/−7 mg (SEM, n=10) vs a post med leak of 110 mg+/−53 mg. This however was less statistically significant (P=0.24). Some subjects in this H-2 group did not show a significant leak increase. Accordingly, an H-2 blocker-induced leak might have a tighter time course than a PPI-induced leak.

Therefore, a second H-2 blocker study was performed looking at leak in an n=5 group but doing evening doses of famotidine and maintaining the other aspects of the above study, with the post H-2 blocker leak test performed again the night of the 5th dose, but now only with a 4-6 hours after the 5th and final dose.

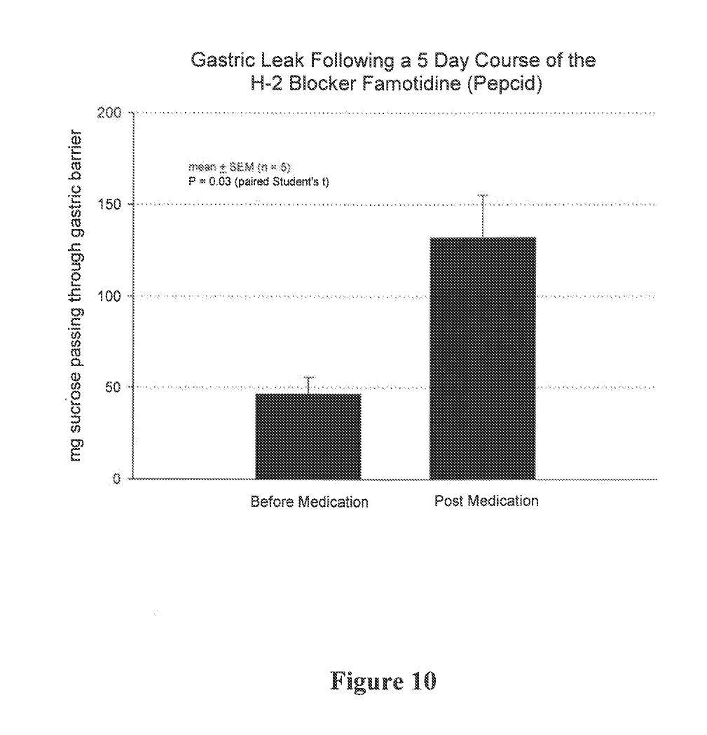

In the second study, the pre H-2 blocker leak was 46 mg+/−9 mg (SEM, n=5). The post H-2 blocker leak was now 132 mg+/−23 mg (FIG. 10). This difference is statistically significant (P=0.03, paired t test). Accordingly, H2 blockers induce a GI leak similar to the leak observed with PPI.

### Example 5

### PPI Induced Gastric Leak Allows Passage of Biologically Relevant Peptide

Omeprazole causes increased leak of bradykinin. Bradykinin is a nonapeptide (Arg-Pro-Pro-Gly-Phe-Ser-Pro-Phe-Arg; SEQ ID NO: 1) that causes blood vessels to dilate, thereby causing blood pressure to lower. Bradykinin has a molecular weight of approximately 1 kD and is hydrophilic. However, a similar increase in oxytocin leak over control was not observed. Oxytocin is a mammalian hormone which also acts as a neurotransmitter in the brain. Oxytocin is also a nonapeptide (Cys-Tyr-Iso-Gln-Asn-Cys-Pro-Leu-Gly; SEQ ID NO: 2), comprises a sulfur bridge between the two cysteines, and has a molecular mass of approximately 1 kD. Without being bound by theory, the oxytocin peptide likely failed to pass through the PPI induced leak because of 1) its higher hydrophobicity, 2) a steric hindrance due to the loop formed by the disulfide bridge, and/or 3) the presence of a disulfide bridge.

The numbers provided hereinbelow (Table 6) represent the transepithelial diffusion (mucosal to serosal) of radiolabeled bradykinin. This data has been corrected for any metabolic breakdown of bradykinin that occurred. It includes only intact bradykinin.

Two data sets from two experiments are provided. These are bradykinin fluxes in picomoles/min/cm². One tissue was treated only with DMSO (vehicle control). It shows a small increase in flux. The other tissue (paired pieces of rat gastric corpus) is treated with omeprazole in DMSO. It shows a much greater increase. The bradykinin leak in controls is likely due to ‘experimental artifact’—in this case mechanical damage of the tissue in handling plus incubation. The increase in flux (leak) of bradykinin seen with DMSO may not be a function of the DMSO at all, but instead simply be a result of tissue ‘breakdown’ over time due to the tissue's very finite lifespan once out of the animal.

TABLE 6
Bradykinin flux values (in pmoles/min/cm²) corrected after TLC
Experiment 1:

| | | | |
|---|---|---|---|
| Pre-DMSO = | 11.3 | Pre-Omep = | 14.5 |
| Post-DMSO = | 13.7 | Post-Omep = | 23.8 |
| % Increase: | 21% | % increase: | 64% |

Experiment 2:

| | | | |
|---|---|---|---|
| Pre-DMSO = | 3.5 | Pre-Omep = | 3.5 |
| Post-DMSO = | 5.0 | Post-Omep = | 9.7 |
| % increase: | 43% | % increase: | 177% |

Average increase with DMSO: 32% (range 21% to 43%)
Average increase with PPI: 120% (range 64% to 177%)

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of orally administering at least one therapeutic agent to a patient in need thereof, said method comprising
a) orally administering to said patient at least one composition comprising at least one gastrointestinal leak inducing compound selected from the group consisting of proton pump inhibitor and H₂ receptor antagonist, and at least one pharmaceutically acceptable carrier, and
b) orally administering to said patient at least one composition comprising said therapeutic agent and at least one pharmaceutically acceptable carrier, wherein said gastrointestinal leak inducing compound is administered prior to said therapeutic agent.

2. The method of claim 1, wherein said gastrointestinal leak inducing compound is a proton pump inhibitor.

3. The method of claim 2, wherein said proton pump inhibitor is selected from the group consisting of omeprazole, esomeprazole, lansoprazole, pantoprazole, rabeprazole, pariprazole, leminoprazole, timoprazole, tenatoprazole, disulprazole, RO 18-5362, and IY 81149.

4. The method of claim 3, wherein said proton pump inhibitor is omeprazole or esomeprazole.

5. The method of claim 1, wherein said therapeutic agent is delivered to the blood stream after said oral administration.

6. The method of claim 1, wherein said therapeutic agent is selected from the group consisting of peptide, protein, an antibody, nucleic acid molecule, an oligonucleotide, a chemical compound, and a small molecule.

7. The method of claim 1, wherein said therapeutic agent is high molecular weight.

8. The method of claim 1, wherein said therapeutic agent is charged.

9. The method of claim 1, wherein said therapeutic agent is hydrophilic.

10. The method of claim 1, wherein said therapeutic agent is not specifically transported transcellularly.

11. The method of claim 1, with the proviso that an acid secretagogue is not co-administered to said patient.

12. The method of claim 1, wherein said therapeutic agent is not indicated for oral administration.

13. The method of claim 1, wherein said therapeutic agent is indicated for intravenous administration or administration by injection.

14. The method of claim 1, wherein said gastrointestinal leak inducing compound is administered at least one day before the administration of said therapeutic agent.

15. The method of claim 14, wherein said gastrointestinal leak inducing compound is administered at least three days before the administration of said therapeutic agent.

16. The method of claim 15, wherein said gastrointestinal leak inducing compound is administered at least five days before the administration of said therapeutic agent.
